(19) United States
(12) Patent Application Publication
(10) Pub. No.: US 2026/0066087 A1
(43) Pub. Date: Mar. 5, 2026

(54) TECHNIQUES FOR PREDICTING AND TREATING POST-OPERATIVE OUTCOMES IN SURGERY PATIENTS

(71) Applicant: UNIVERSITY OF FLORIDA RESEARCH FOUNDATION, INCORPORATED, Gainesville, FL (US)

(72) Inventor: Ken PORCHE, Gainesville, FL (US)

(21) Appl. No.: 19/107,233

(22) PCT Filed: Sep. 8, 2023
(86) PCT No.: PCT/US2023/032314

§ 371 Date: Feb. 27, 2025

Related U.S. Application Data
(60) Provisional application No. 63/404,721, filed on Sep. 8, 2022.

(51) Int. Cl. G16H 20/40; A61B 5/20; G06N 3/04

(52) U.S. Cl. CPC G16H 20/40; A61B 5/202; G06N 3/04

(57) ABSTRACT

Techniques for treating a subject undergoing spinal surgery include obtaining first data that indicates demographic, medical history or surgery information. A first probability for the subject developing post-operative urinary retention (POUR) is generated by inputting the first data into an input layer of a neural network trained with training data that indicates corresponding information for retrospective patients of spinal surgery and POUR outcomes for those patients. A signal is sent, which indicates a POUR classification for the subject based at least in part on the first probability. The subject is then treated based at least in part on the signal. A binomial regression models trained on a subset of training data is used optionally to produce a second probability. Optionally, the signal indicates a classification based on first or second cutoffs for the two probabilities.

### BACKGROUND

A substantial number of patients are observed to develop post operative complications after surgery, such as post-operative urinary retention (POUR) after spinal surgery. POUR outcomes after lower spinal surgery can be as high as 30%. POUR contributes to increased hospital stays, worse outcomes for patients, and costly bladder interventions.

A subject includes a human or other animal patient or study participant undergoing surgery or any portion of the surgical procedure.

Treatment includes any action or action avoidance or change to mediate the post-surgical outcome. For example, treatment of POUR includes catheterization or urinary function tests, and medication administration. This can be uncomfortable for the patient and costly for the hospital.

### SUMMARY

Techniques are provided for predicting and treating post-operative outcomes in surgery patients. Machine learning based on retrospective outcomes is used including one or more of a multiple regression model and neural network model, which can be stacked together to classify a subject and treat the subject accordingly.

In a first set of embodiments, a method for treating a subject undergoing surgery includes obtaining, on a processor, first data for a subject undergoing surgery. The first data indicates demographic information for the subject or medical history for the subject or surgery information about the surgery, the latter optionally indicating any anesthesia administered, or some combination. The method also includes generating, on the processor, neural network output data that indicates a first probability for the subject developing a post-operative outcome by inputting the first data into an input layer of a neural network. The neural network is trained with training data that indicates, for a retrospective plurality of prior patients of surgery, demographic information for the retrospective plurality or medical history for the retrospective plurality or surgery information about surgeries for the retrospective plurality, or some combination. The training data, unlike the first data, includes post-operative outcomes for the retrospective plurality. Still further, the method includes sending from the processor a signal that indicates a post-operative outcome classification for the subject based at least in part on the neural network output data. Even further still, the method includes treating the subject based at least in part on the signal.

In some embodiments of the first set, the signal indicates that the subject should be treated for the post-operative outcome when the first probability exceeds a first cutoff value.

In some embodiments of the first set, the first data includes values for over 200 parameters. In some of these embodiments, inputting the first data into the input layer of the neural network includes scaling the first data with a scaling factor for each parameter such that all values of that parameter for the training data lie in a range from 0 to 1 inclusive.

In some embodiments of the first set, the neural network includes two hidden layers, each hidden layer fully connected to a preceding layer and each hidden layer using a sigmoid activation function. In some of these embodiments, a first hidden layer of the two hidden layers is fully connected to the input layer; and the first hidden layer has a first number nodes in a range from 20 to 80. In some of these embodiments, a second hidden layer of the two hidden layers is fully connected to the first hidden layer and the second hidden layer comprises a second number nodes in a range from 10 to 40.

In some embodiments of the first set, an output layer of the neural network comprises one output node that indicates the first probability and uses an identity activation function; and the output layer uses a sum of squares error function during training.

In some embodiments of the first set, the method also includes generating, on the processor, binomial regression output data that indicates a second probability for the subject developing POUR by inputting a small subset of the first data into an input layer of a binomial regression trained with a corresponding subset of the training data. Here, the POUR classification for the subject is further based at least in part on the binomial regression output data. In some of these embodiments, the signal indicates that the subject should be treated for POUR when the first probability exceeds the first cutoff value OR when the second probability exceeds the second cutoff value. Alternatively, the signal indicates that the subject should be treated for POUR when the first probability exceeds the first cutoff value AND when the second probability exceeds the second cutoff value.

In some embodiments of the first set that use the binomial regression, the small subset includes values for fewer than 50 parameters. For example, in some embodiments, the small subset includes only subset parameters of the first data, wherein the subset parameters are correlated with the POUR outcomes for the training set with a p value less than threshold significance level. In some of these embodiments, the threshold significance level is a p value less than 0.05.

In some embodiments of the first set, the surgery is spinal surgery and the outcome is a post-operative urine retention (POUR) outcome. In some of these embodiments, treating the subject includes use or avoidance of anesthetic agents, use or avoidance of analgesic medications, indwelling catheter placement, or surgical choice.

In other sets of embodiments, a non-transient computer-readable medium or an apparatus or a neural network is configured to perform one or more steps of the above methods.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1 is a block diagram that illustrates an example training set for machine learning of POUR outcomes, according to an embodiment;

FIG. 2 is a flow chart that illustrates an example method for machine learning based on a training set, according to an embodiment;

FIG. 3A is a block diagram that illustrates an example neural network for illustration;

FIG. 3B is a plot that illustrates example activation functions used to combine inputs at any node of a feed forward neural network, according to various embodiments;

FIG. 4 is a flow chart that illustrates an example method to treat a subject undergoing spinal surgery based on machine learning, according to an embodiment;

FIG. 5A is a plot that illustrates example classification performance based on a cutoff applied to model output, according to an embodiment;

FIG. 5B is a flow chart that illustrates an example method to stack two machine learning models to classify a particular subject, according to an embodiment;

FIG. 5C is a plot that illustrates an example performance when stacking two machine learning models to classify a particular subject, according to an embodiment;

FIG. 6 is a block diagram that illustrates a computer system upon which an embodiment of the invention may be implemented;

FIG. 7 illustrates a chip set upon which an embodiment of the invention may be implemented; and

FIG. 8 is a diagram of exemplary components of a mobile terminal (e.g., cell phone handset) for communications, which is capable of operating in the system, according to one embodiment.

FIG. 9 Bar graph of the differences in rates of POUR based on patient demographics and medication use of the overall cohort (n=891). Frequencies (N) and p values comparing those who did and did not develop urinary retention are listed in the label. Red bars=included in binomial logistic regression and neural network models. Blue bars=only included in neural network model. Figure is available in color online only.

FIG. 10 Bar graph of the differences in rates of POUR based on planned surgical characteristics of the overall cohort (n=891). Frequencies of the surgery type (N) and p values comparing those who did and did not develop urinary retention are listed in the label. Red bars=included in binomial logistic regression and neural network models. Blue bars=only included in neural network model. Figure is available in color online only.

FIG. 11A-B Scatterplot for the testing set (N=235) of the predicted probabilities of the regression model (y-axis) compared to the neural network model (x-axis) demonstrating (FIG. 11A) optimal cutoff points of 0.43 and 0.54, respectively, and (FIG. 11B) optimal cutoff points of 0.23 and 0.24, respectively, for combining the two models. By combining the two models using an “and” conjunction, the upper right quadrant (red shaded area) represents a positive test result. Thus, a green diamond in this area represents a true positive, whereas a blue circle in this area represents a false positive. A green or blue marker in the other quadrants represents a false negative or a true positive, respectively. Figure is available in color online only.

FIG. 12 Bar graph of the differences in rates of POUR based on medical comorbidities (pre-existing ICD 10 codes) of the overall cohort (n=891). Frequencies of the ICD 10 code (N) and p-values comparing those who did and did not develop urinary retention are listed in the label. All codes included here have an association with POUR with p<0.0003. Solid=included in neural network model only. Dotted=included in regression and neural network models.

FIG. 13 Bar graph of the differences in rates of POUR based on medical comorbidities (pre-existing ICD 10 codes) of the overall cohort (n=891). Frequencies of the ICD 10 code (N) and p-values comparing those who did and did not develop urinary retention are listed in the label. All codes included here have an association with POUR with p>0.0003.

### DETAILED DESCRIPTION

A method and apparatus are described for predicting and treating post-operative outcomes such as urinary retention (POUR) in spinal surgery patients. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of a particular regression model and a two-hidden-layer neural network used in combination for dealing with POUR outcomes after spinal surgery. However, the invention is not limited to this context. In other embodiments, either model is used separately, or other regression model classifiers or other neural networks are used in combination to predict and treat other outcomes of other surgeries. This can apply broadly to neurosurgical, general, and thoracic surgery procedures where extended case time is often seen.

### 1. OVERVIEW

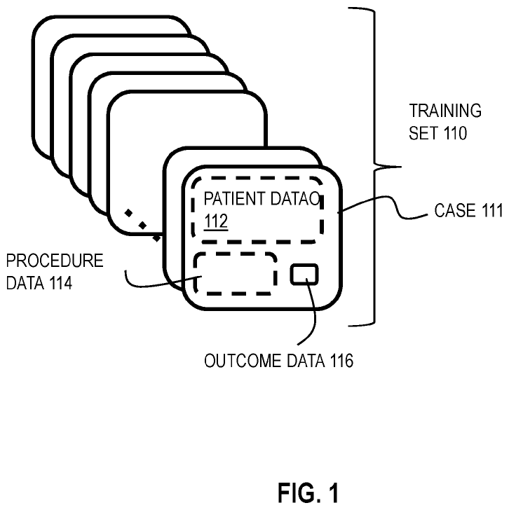

FIG. 1 is a block diagram that illustrates an example training set 110 for machine learning of procedure outcomes, according to an embodiment. The training set 110 includes multiple retrospective cases, such as case 111, for which outcomes of interest are known or can be determined. The cases 111 for the training set 110 are selected to be appropriate for the population of interest, e.g., for surgeries, such as spinal surgery, or more specifically, lower spinal surgeries, that can lead to POUR or other outcomes of interest. The training set is in machine readable form, such as a data structure or signal on a computer readable medium. Each case 111 includes patient data 112 indicating information 112 about a retrospective patient and procedure data 114 indicating information about a procedure as well as outcome data 116 indicating information about the outcome of interest. The patient data 112 includes multiple fields that hold data that indicate demographic (e.g., age, height, weight, allergies) and medical history (e.g., heart disease, diabetes, former orthopedic injuries) about the retrospective subject. Much of this is available as digital medical records and is dominated by HIPPA security requirements. Thus, in some embodiments identifying information about the retrospective patient is omitted from the patient data 112. The procedure data 114 includes multiple fields that hold data that indicate information about the retrospective procedures (e.g., anatomical targets, locations and sequence of incisions, tools used, prosthetics, implants, medications and anesthesia administered). In some embodiments, this information is represented in whole or in part by insurance codes: lumbar discectomy (CPT codes 63030 or 63035), lumbar laminectomy (CPT codes 63005, 63012, 63017, 63042, 63047, or 64048), single-level lumbar fusion (CPT codes 22633), multi-level lumbar fusion (CPT codes 22534, 22585, 22614, 22632, or 22634), interbody fusion (CPT code 22851), anterior or lateral interbody fusion (CPT codes 22845-22847), posterior interbody fusion (CPT codes 22840 or 22842-22844). The outcome data 114 includes multiple fields that hold data that indicate information about the outcome (e.g., function recovered, infections, POUR).

The models can be developed using codes based on the International Classification of Disease (ICD) adapted for the Health Insurance Portability and Accountability Act of 1996 (HIPAA), currently at version 10 (https://icd.codes/) and Current Procedural Terminology® (CPT®), set by the American Medical Association, updated on a rolling basis (www.ama-assn.org/amaone/cpt-current-procedural-terminology). In some embodiments, the training data, validation data, test data or comprises inputs directed to at least one international classification of diseases (ICD) code or CPT® code. These and other variables are provided in Table 1 below:

TABLE 1

| | | Inclusion in | Inclusion in |
|---|---|---|---|
| | | regression | neural network |
| Variables | | model | model |
| N = 175 | Variable type | (N = 22) | (N = 174) |
| Preoperative (N = 12) | | | |
| Age | Continuous | X | X |
| BMI | Continuous | X | X |
| Any preoperative opioid use | Binary (0/1) | X | |
| Preoperative oxycodone use | Binary (0/1) | | X |
| Preoperative fentanyl use | Binary (0/1) | | X |
| Preoperative morphine use | Binary (0/1) | | X |
| Preoperative hydromorphone use | Binary (0/1) | | X |
| Preoperative methadone use | Binary (0/1) | | X |
| Preoperative meperidine use | Binary (0/1) | | X |
| Preoperative tamsulosin use | Binary (0/1) | | X |
| Preoperative doxazosin use | Binary (0/1) | | X |
| Sex | Binary (1/2) | | X |
| Surgical characteristics (N = 17) | | | |
| Number of Levels | Continuous | | X |
| Laminectomy | Binary (0/1) | X | X |
| Single Laminectomy | Binary (0/1) | X | X |
| Discectomy | Binary (0/1) | | X |
| Fusion | Binary (0/1) | | X |
| Single level Fusion | Binary (0/1) | | X |
| Multi-level Fusion | Binary (0/1) | | X |
| Interbody fusion type: | | | |
| Transforaminal | Binary (0/1) | | X |
| Lateral | Binary (0/1) | | X |
| Anterior | Binary (0/1) | | X |
| Pelvic screw placement | Binary (0/1) | | X |
| Minimally invasive approach | Binary (0/1) | | X |
| Multiple Laminectomies | Binary (0/1) | | X |
| Posterolateral fusion only | Binary (0/1) | | X |
| Discectomy only | Binary (0/1) | | X |
| Laminectomy only | Binary (0/1) | | X |
| Single-level interbody fusion | Binary (0/1) | | X |
| ICD-10 codes (comorbidities) (N = 146) | | | |
| Z981 | Binary (0/1) | X | X |
| Z98890 | Binary (0/1) | X | X |
| M4316 | Binary (0/1) | X | X |
| R000 | Binary (0/1) | X | X |
| I517 | Binary (0/1) | X | X |
| E119 | Binary (0/1) | X | X |
| Z01818 | Binary (0/1) | X | X |
| I959 | Binary (0/1) | X | X |
| R6889 | Binary (0/1) | X | X |
| K6389 | Binary (0/1) | X | X |
| Z136 | Binary (0/1) | X | X |
| R52 | Binary (0/1) | X | X |
| K5900 | Binary (0/1) | X | X |
| N390 | Binary (0/1) | X | X |
| K567 | Binary (0/1) | X | X |
| R4182 | Binary (0/1) | X | X |
| R339 | Binary (0/1) | X | X |
| Z4789 | Binary (0/1) | | X |
| M48061 | Binary (0/1) | | X |
| I10 | Binary (0/1) | | X |
| M4802 | Binary (0/1) | | X |
| R918 | Binary (0/1) | | X |
| M48062 | Binary (0/1) | | X |
| J9811 | Binary (0/1) | | X |
| R9431 | Binary (0/1) | | X |
| M5126 | Binary (0/1) | | X |
| M5416 | Binary (0/1) | | X |
| G8918 | Binary (0/1) | | X |
| Z09 | Binary (0/1) | | X |
| M5116 | Binary (0/1) | | X |
| D62 | Binary (0/1) | | X |
| G8929 | Binary (0/1) | | X |
| J90 | Binary (0/1) | | X |
| R509 | Binary (0/1) | | X |
| M47816 | Binary (0/1) | | X |
| M545 | Binary (0/1) | | X |
| J984 | Binary (0/1) | | X |
| M7989 | Binary (0/1) | | X |
| Z452 | Binary (0/1) | | X |
| E785 | Binary (0/1) | | X |
| M5136 | Binary (0/1) | | X |
| T1490XA | Binary (0/1) | | X |
| Z4682 | Binary (0/1) | | X |
| G8911 | Binary (0/1) | | X |
| T148XXA | Binary (0/1) | | X |
| M47812 | Binary (0/1) | | X |
| D649 | Binary (0/1) | | X |
| M4806 | Binary (0/1) | | X |
| R531 | Binary (0/1) | | X |
| M4804 | Binary (0/1) | | X |
| I2510 | Binary (0/1) | | X |
| W19XXXA | Binary (0/1) | | X |
| Z5189 | Binary (0/1) | | X |
| R079 | Binary (0/1) | | X |
| V892XXA | Binary (0/1) | | X |
| I4891 | Binary (0/1) | | X |
| R140 | Binary (0/1) | | X |
| Z4659 | Binary (0/1) | | X |
| G9520 | Binary (0/1) | | X |
| Z01810 | Binary (0/1) | | X |
| M960 | Binary (0/1) | | X |
| M549 | Binary (0/1) | | X |
| M4317 | Binary (0/1) | | X |
| R0689 | Binary (0/1) | | X |
| M5442 | Binary (0/1) | | X |
| D72829 | Binary (0/1) | | X |
| M5441 | Binary (0/1) | | X |
| M5117 | Binary (0/1) | | X |
| Z9911 | Binary (0/1) | | X |
| R200 | Binary (0/1) | | X |
| M4312 | Binary (0/1) | | X |
| R0902 | Binary (0/1) | | X |
| M961 | Binary (0/1) | | X |
| M5127 | Binary (0/1) | | X |
| E039 | Binary (0/1) | | X |
| G959 | Binary (0/1) | | X |
| N179 | Binary (0/1) | | X |
| R600 | Binary (0/1) | | X |
| K219 | Binary (0/1) | | X |
| G061 | Binary (0/1) | | X |
| Z86718 | Binary (0/1) | | X |
| M419 | Binary (0/1) | | X |
| J811 | Binary (0/1) | | X |
| X58XXXA | Binary (0/1) | | X |
| E878 | Binary (0/1) | | X |
| Z1389 | Binary (0/1) | | X |
| A419 | Binary (0/1) | | X |
| R0602 | Binary (0/1) | | X |
| M542 | Binary (0/1) | | X |
| E871 | Binary (0/1) | | X |
| J189 | Binary (0/1) | | X |
| E46 | Binary (0/1) | | X |
| J939 | Binary (0/1) | | X |
| M4807 | Binary (0/1) | | X |
| J449 | Binary (0/1) | | X |
| M5410 | Binary (0/1) | | X |
| R937 | Binary (0/1) | | X |
| M4726 | Binary (0/1) | | X |
| E1165 | Binary (0/1) | | X |
| R7881 | Binary (0/1) | | X |
| G9589 | Binary (0/1) | | X |
| R410 | Binary (0/1) | | X |
| E669 | Binary (0/1) | | X |
| R109 | Binary (0/1) | | X |
| V877XXA | Binary (0/1) | | X |
| M2578 | Binary (0/1) | | X |
| I38 | Binary (0/1) | | X |
| R001 | Binary (0/1) | | X |
| F329 | Binary (0/1) | | X |
| G062 | Binary (0/1) | | X |
| J9601 | Binary (0/1) | | X |
| M4186 | Binary (0/1) | | X |
| M4626 | Binary (0/1) | | X |
| S32009K | Binary (0/1) | | X |
| R011 | Binary (0/1) | | X |
| R933 | Binary (0/1) | | X |
| S0003XA | Binary (0/1) | | X |
| Z4889 | Binary (0/1) | | X |
| M9983 | Binary (0/1) | | X |
| S32019A | Binary (0/1) | | X |
| Z930 | Binary (0/1) | | X |
| S12600A | Binary (0/1) | | X |
| M79605 | Binary (0/1) | | X |
| R202 | Binary (0/1) | | X |
| M4800 | Binary (0/1) | | X |
| F419 | Binary (0/1) | | X |
| M869 | Binary (0/1) | | X |
| N189 | Binary (0/1) | | X |
| M5137 | Binary (0/1) | | X |
| G9529 | Binary (0/1) | | X |
| E876 | Binary (0/1) | | X |
| G834 | Binary (0/1) | | X |
| I509 | Binary (0/1) | | X |
| M4624 | Binary (0/1) | | X |
| M5124 | Binary (0/1) | | X |
| D696 | Binary (0/1) | | X |
| S32018A | Binary (0/1) | | X |
| M5417 | Binary (0/1) | | X |
| T84216A | Binary (0/1) | | X |
| R739 | Binary (0/1) | | X |
| S2242XA | Binary (0/1) | | X |
| I471 | Binary (0/1) | | X |
| C7951 | Binary (0/1) | | X |
| M8448XA | Binary (0/1) | | X |
| G9389 | Binary (0/1) | | X |
| R609 | Binary (0/1) | | X |

In some embodiments, other information about the subject is included for both the training set and the during operation of the models. For example, one or more biomarkers can be included in the set of parameters. As used herein, the term “biomarker” (or fragment thereof, or variant thereof) and their synonyms, which are used interchangeably, refer to molecules that can be evaluated in a sample and are associated with a physical condition. For example, markers include expressed genes, their transcripts (e.g. mRNA) or their products (e.g., proteins) or autoantibodies to those proteins that can be detected from human samples, such as blood, serum, solid tissue, and the like, that is associated with a physical or disease condition. Such biomarkers include, but are not limited to, biomolecules comprising nucleotides, amino acids, sugars, fatty acids, steroids, metabolites, polypeptides, proteins (such as, but not limited to, antigens and antibodies), carbohydrates, lipids, hormones, antibodies, regions of interest which serve as surrogates for biological molecules, combinations thereof (e.g., glycoproteins, ribonucleoproteins, lipoproteins) and any complexes involving any such biomolecules, such as, but not limited to, a complex formed between an antigen and an autoantibody that binds to an available epitope on said antigen. In a specific embodiment, the biomarker is an expression product of a gene.

The term “biomarker value” refers to a value measured or derived for at least one corresponding biomarker of the subject and which is typically at least partially indicative of a concentration of the biomarker in a sample taken from the subject. Thus, the biomarker values could be measured biomarker values, which are values of biomarkers measured for the subject, or alternatively could be derived biomarker values, which are values that have been derived from one or more measured biomarker values, for example by applying a function to the one or more measured biomarker values. Biomarker values can be of any appropriate form depending on the manner in which the values are determined. For example, the biomarker values could be determined using high-throughput technologies such as mass spectrometry, sequencing platforms, array and hybridization platforms, immunoassays, flow cytometry, or any combination of such technologies and in one preferred example, the biomarker values relate to a level of activity or abundance of an expression product or other measurable molecule, quantified using a technique such as PCR, sequencing or the like. In this case, the biomarker values can be in the form of amplification amounts, or cycle times, which are a logarithmic representation of the concentration of the biomarker within a sample, as will be appreciated by persons skilled in the art and as will be described in more detail below. The term “expression product” refers to a polynucleotide expression product (e.g., transcript) or a polypeptide expression product (e.g., protein)

In the illustrated embodiment, spinal surgeries and POUR outcomes are of interest and an example training set is described in the Appendix. For example, the anonymous fields specifying retrospective patient data 112, and codes specifying retrospective procedure data 114, and representations for POUR outcomes are described in more detail in the Appendix.

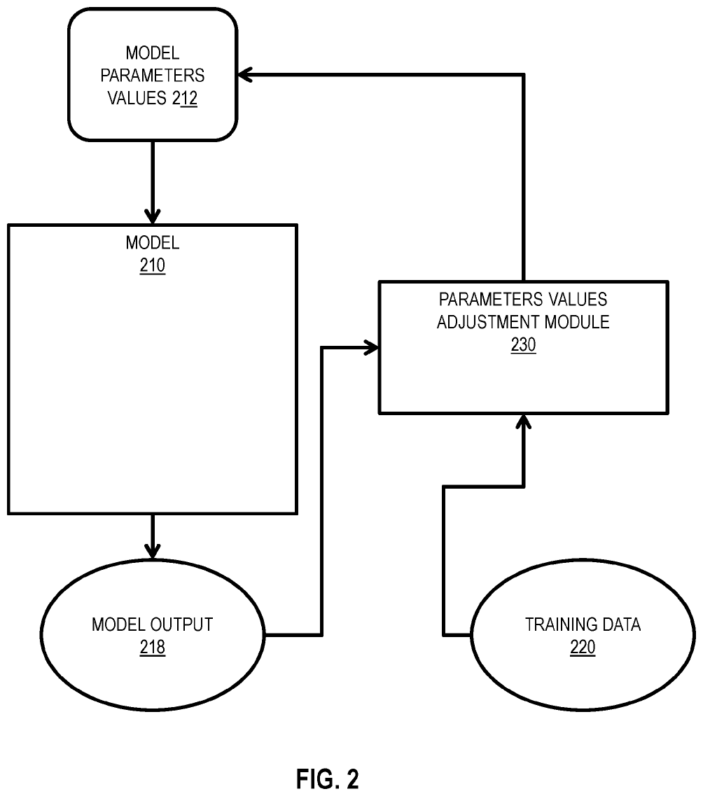

FIG. 2 is a flow chart that illustrates an example method for machine learning based on a training set, according to an embodiment. Many machine methods to fit models using training sets are well known and are available as commercial software such as MATLAB™ from MATHWORKS of Natick, Massachusetts. First one sets a function that takes in values for a set of parameters and returns a model output data set. Second one sets an ‘error function’ that provides a number representing the difference between the training set data and the model's output for any given set of values for the model parameters. This is usually either the sums of squared error (SSE) or maximum likelihood. Third one determines the parameter values that minimize this difference. The model 210 is the function that takes in a set of parameter values. There is a practically endless number of model and functions that can be fit to data. In the illustrated embodiments a binomial classifier is used a one model and a neural network is used as an independent model. The training data is represented by oval 220 and the model output is represented by oval 218. The definition of the error function and the process of modifying the parameter values to reduce the error at each iteration is represented by box 230. And the full effort to minimize the error occurs by repeating this cycle until the errors are low enough to meet some criterion.

In some embodiments a validation set is used in which the outcome is known but the validation set is not used to train the model 210. Instead, the validation set is used to determine how well the trained model fits new data. This is done to establish confidence and estimate error rates for the model. If errors are small enough and confidence is high enough, the trained model or models are used during operations on subjects with unknown outcomes.

In an example embodiment, a binomial logistic model-which estimates the probability that an outcome is present given the values of explanatory variables and is typically used for classification—was formed with backward elimination based on significant changes in likelihood ratios, using a 0.10 cutoff. All patient demographics and surgical characteristics were considered for the model, but only comorbidities that had significant correlations with POUR were included (p<0.05 corrected for multiple comparisons). For example, the inputs used in developing the predictive POUR model may involve at least one ICD code, at least two ICD codes, at least five ICD codes, at least 10 ICD codes, or between 1 and 10 ICD codes shown to be associated with higher risk of POUR. Example ICD codes associated with POUR and used for formation of the predictive model include, but are not limited to, diabetes (ICD code E11.9), abnormal heartbeat (ICD code R00), other general symptoms and signs (ICD code R68.89), altered mental status (ICD code R41.82), screening for cardiovascular disorders (ICD code Z13.6) and code for plans for only a single-level laminectomy (ICD code M96.1). These comorbidities were derived from associations discovering in the training set. This resulted in fewer than 50 inputs. For example, in an example embodiment described in the Appendix about 26 inputs are used.

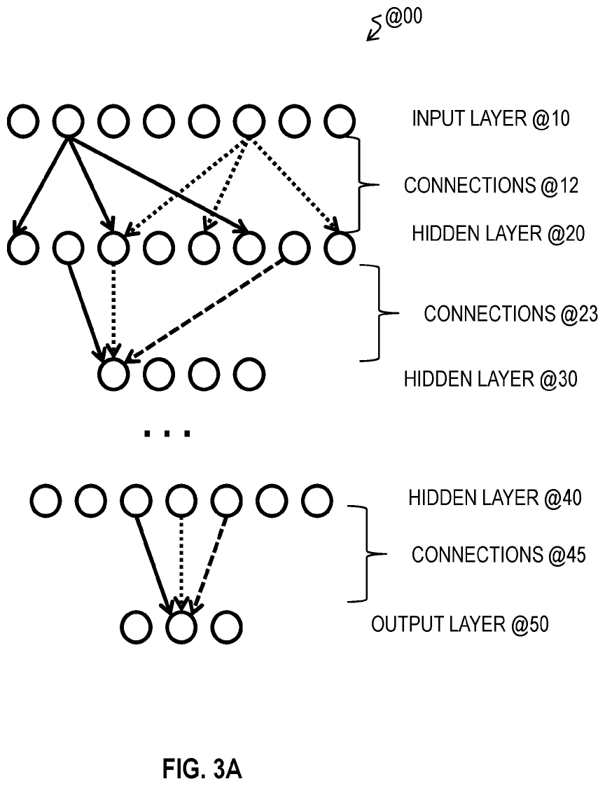

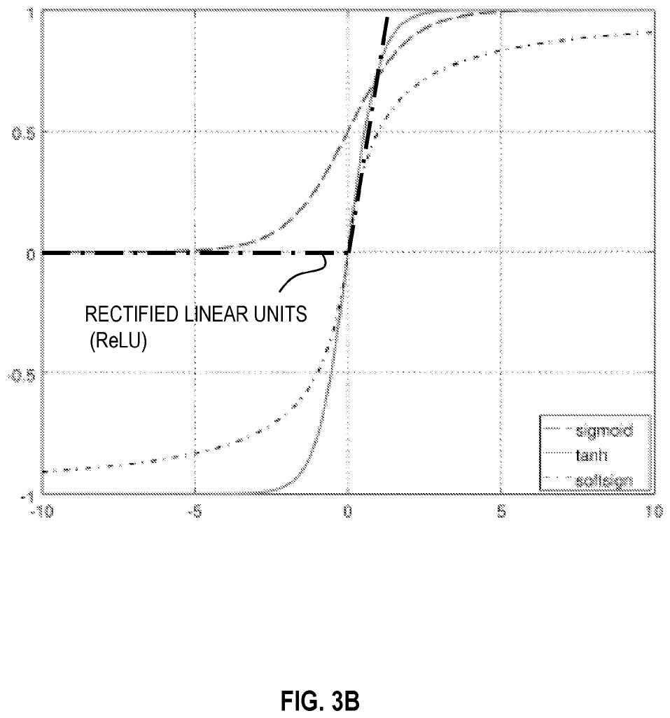

Effective training of a machine learning system with the characteristics described above can be achieved using neural networks, widely used in image processing and natural language processing. FIG. 3A is a block diagram that illustrates an example neural network 300 for illustration. A neural network 300 is a computational system, implemented on a general-purpose computer, or field programmable gate array, or some application specific integrated circuit (ASIC), or some neural network development platform, or specific neural network hardware, or some combination. The neural network is made up of an input layer 310 of nodes, at least one hidden layer 320, 330 or 340 of nodes, and an output layer 350 of one or more nodes. Each node is an element, such as a register or memory location, that holds data that indicates a value. The value can be code, binary, integer, floating point, or any other means of representing data. Values in nodes in each successive layer after the input layer in the direction toward the output layer is based on the values of one or more nodes in the previous layer. The nodes in one layer that contribute to the next layer are said to be connected to the node in the later layer. Connections 312, 323, 345 are depicted in FIG. 3A as arrows. The values of the connected nodes are combined at the node in the later layer using some activation function with scale and bias (also called weights) that can be different for each connection. Neural networks are so named because they are modeled after the way neuron cells are connected in biological systems. A fully connected neural network has every node at each layer connected to every node at any previous or later layer.

FIG. 3B is a plot that illustrates example activation functions used to combine inputs at any node of a neural network. These activation functions are normalized to have a magnitude of 1 and a bias of zero; but when associated with any connection can have a variable magnitude given by a weight and centered on a different value given by a bias. The values in the output layer 350 depend on the values in the input layer and the activation functions used at each node and the weights and biases associated with each connection that terminates on that node. The sigmoid activation function (dashed trace) has the properties that values much less than the center value do not contribute to the combination (a so called switch off effect) and large values do not contribute more than the maximum value to the combination (a so called saturation effect), both properties frequently observed in natural neurons. The tanh activation function (solid trace) has similar properties but allows both positive and negative contributions. The softsign activation function (short dash-dot trace) is similar to the tanh function but has much more gradual switch and saturation responses. The rectified linear units (ReLU) activation function (long dash-dot trace) simply ignores negative contributions from nodes on the previous layer; but, increases linearly with positive contributions from the nodes on the previous layer; thus, ReLU activation exhibits switching but does not exhibit saturation. The identity activation function applies identity operation on input data so output data is proportional to the input data; thus, it exhibits neither switching nor saturation effects. In some embodiments, the activation function operates on individual connections before a subsequent operation, such as summation or multiplication; in other embodiments, the activation function operates on the sum or product of the values in the connected nodes. In other embodiments, other activation functions are used, such as kernel convolution.

An advantage of neural networks is that they can be trained to produce a desired output from a given input without knowledge of how the desired output is computed. There are various algorithms known in the art to train the neural network on example inputs with known outputs. Typically, the activation function for each node or layer of nodes is predetermined, and the training determines the weights and biases for each connection. A trained network that provides useful results, e.g., with demonstrated good performance for known results, is then used in operation on new input data not used to train or validate the network.

In some neural networks, the activation functions, weights and biases are shared for an entire layer. This provides the networks with shift and rotation invariant responses. The hidden layers can also consist of convolutional layers, pooling layers, fully connected layers, and normalization layers. The convolutional layer has parameters made up of a set of learnable filters (or kernels), which have a small receptive field. In a pooling layer, the activation functions perform a form of non-linear down-sampling, e.g., producing one node with a single value to represent four nodes in a previous layer. There are several non-linear functions to implement pooling among which max pooling is the most common. A normalization layer simply rescales the values in a layer to lie between a predetermined minimum value and maximum value, e.g., 0 and 1, respectively.

In some embodiments, a multilayer perceptron (MLP) neural network architecture, as depicted in FIG. 3A, was used because such networks demonstrate an advantageous ability to learn salient features of the data on its own without having to seriously limit the inputs. Thus, all available information can be used. For example, about 174 input parameter values (see Table 1) are used as 345 input layer nodes (171 binary variables equates to 342 nodes and 3 standardized continuous variables which map to one node each), which allows all available patient and procedure information to be used for lower spinal surgery in the retrospective training data. Many of these inputs are true or false represented by the binary values 1 and 0, or −1 and 1, respectively. To keep parameters with inherently large values from dominating the network weights learned during training, it is advantageous, in some embodiments, to scale all the values for each parameter so that observed ranges in the training set for that parameter fall with a limited range, e.g., from 0 to 1, or from

−1 to 1. In the example embodiment, the neural network consisted of two hidden layers terminating at an output layer. The number of nodes were found to optimize the predictive power of the model. A practitioner can determine a number of nodes in each hidden layer by starting with an initial guess for the number of nodes (e.g., based on a number larger than, e.g., twice as large as, the number of parameters expected to be important a priori) in the first layer and something on the order of half that number in the second hidden layer, and again half in each successive layer. The number of nodes in each layer can then be adjusted up or down to see the effect on the performance of the model and stopping when the performance seems to be sufficient for an intended purpose, e.g. to distinguish between those persons to be treated for the outcome and those not treated or treated differently.
The first hidden layer consisted of 38 fully connected nodes. In other embodiments, the first layer is larger to allow other important factors to be discovered or smaller to combine several factors. Thus, a range of node numbers in the first hidden layer is used in various other embodiments, to span the number of the expected factors, wherein the number is selected in a range from about 20 to about 80 nodes. In the example embodiment, the second hidden layer consisted of 21 fully connected nodes. Thus, a range of node numbers in the second hidden layer is used in various other embodiments, to span the number of the expected factors, wherein the number is selected in a range from about 10 to about 40 nodes. In the illustrated embodiment, hidden layers used a sigmoid activation function with no dropout. The output layer used an identity activation function and a sum of squares error function. The stopping rule for training was 1 consecutive step with no decrease in error based on the validation set.

### 2. METHOD

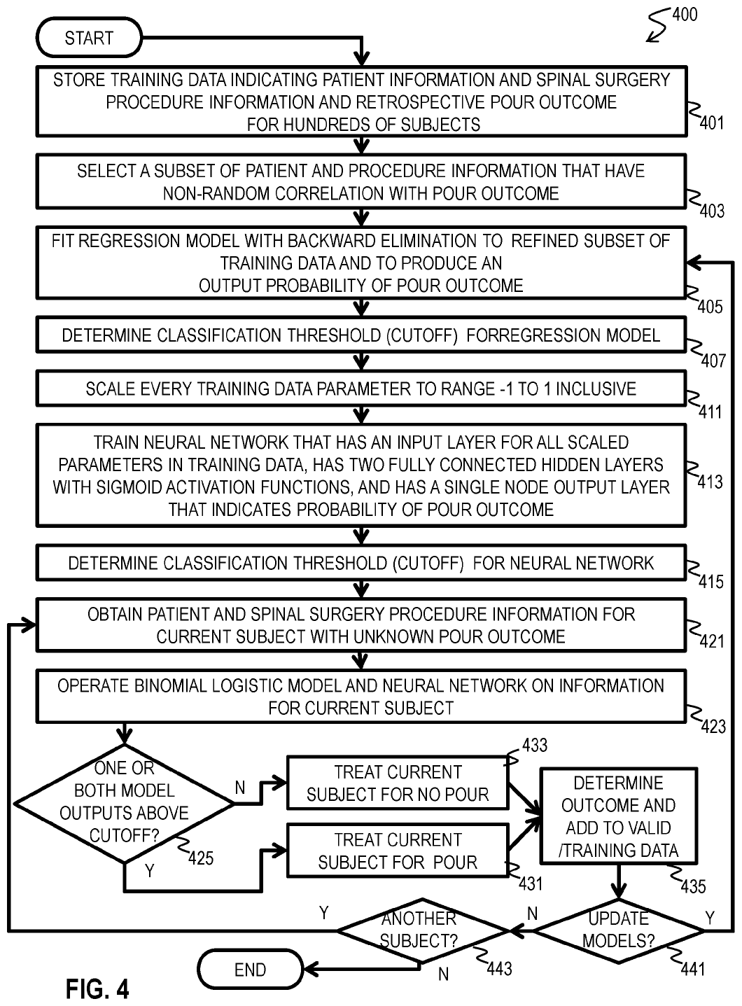

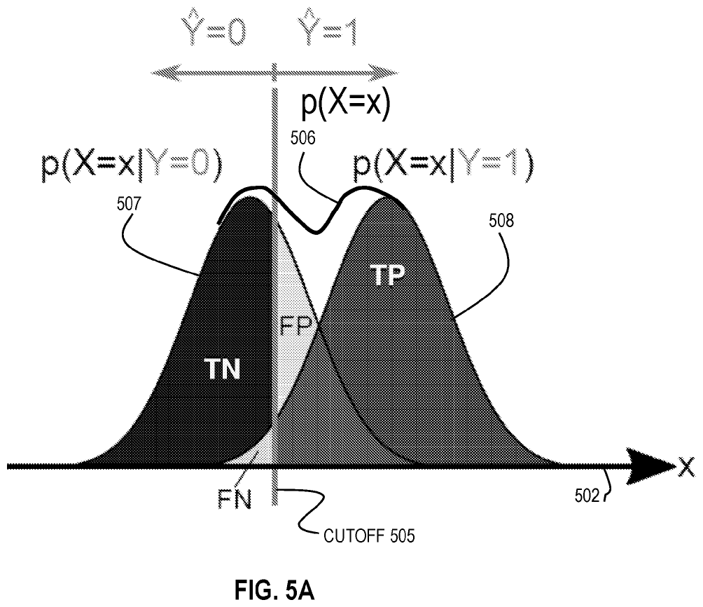

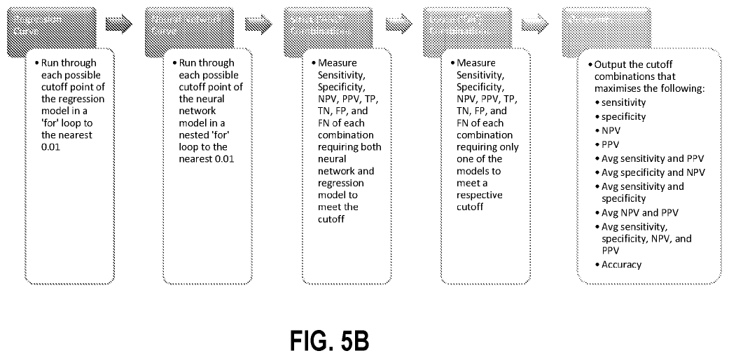

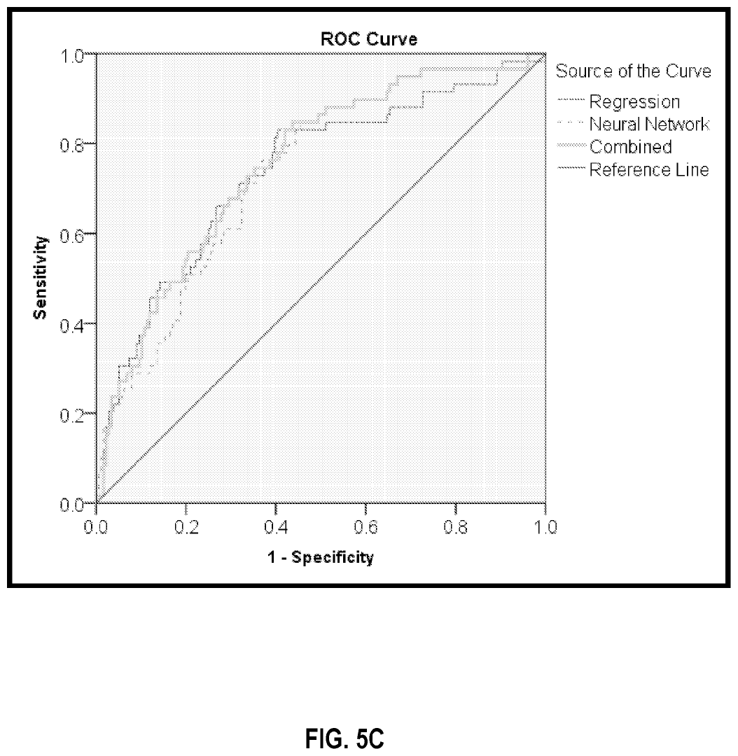

FIG. 4 is a flow chart that illustrates an example method to treat a subject undergoing spinal surgery based on machine learning, according to an embodiment. Although steps are depicted in FIG. 4, and in subsequent flowchart FIG. 5B, as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 401, training data, indicating retrospective outcomes for hundreds of subjects as well as corresponding patient information and procedure information, is obtained and stored on computer readable media. Any method may be used to obtain this information and store it on media, including receiving manual input unsolicited or in response to prompts, e.g., through a graphical user interface (GUI), retrieved in whole or in part from an extant file or database, such as digital medical records and insurance records indicating medical diagnosis and procedure codes, received as network packet traffic either unsolicited or in response to a query message, or some combination. Storing can be accomplished in a local data structure such as a file or database with fields for various parameters indicating patient information, procedure information and outcomes, such as POUR outcomes as depicted in FIG. 1. Thus, step 401 includes obtaining and storing for a retrospective plurality of prior patients of spinal surgery, demographic information for the retrospective plurality or medical history for the retrospective plurality or surgery information about spinal surgeries for the retrospective plurality, or some combination, and POUR outcomes for the retrospective plurality. Training data includes values for most if not all parameters that are to be used in any model predicting outcomes, such as POUR outcomes. For example, POUR was defined according to previous literature as reinsertion of a Foley catheter based on retention urine volume>400 milliliters (mL, 1 mL=10⁻³ liters), or requiring straight catheterization for urine volumes>400 mL. Urine volume was determined per standard of care with nurse-led bladder scanning. The patient characteristics serving as additional parameters included all preexisting ICD-10 codes associated with the patient; age; sex; body mass index (BMI); preoperative opioid use (morphine, methadone, fentanyl, oxycodone, hydrocodone, meperidine); preoperative urinary retention medication use (tamsulosin, doxazosin); and planned or actual surgery specifics. In some embodiments, e.g., using all information available for lower spinal surgery procedures, about 250 parameters are available to various degrees.

In step 403, a subset of patient and procedure information is selected, such that the subset includes demonstrably non-random correlation with the outcome of interest, e.g., the POUR outcome. Any method may be used to select the subset of parameters with non-random correlation to the outcome. For example, the selection can be based on parameters identified in the literature with others added or subtracted based on analysis of all or part of the training data set. In an example embodiment, all patient demographics and surgical characteristics were included in the model, but only patient medical records for comorbidities that had significant correlations with POUR in all or part of the training set were included (p<0.05 corrected for multiple comparisons). In some embodiments, the subset of parameters is determined based on the independently trained neural network described below. For example, of all the input parameters each at a different node, the parameters associated with nodes that have weights less than a threshold weight are considered insignificant contributors and are eliminated from the parameters used with the regression model to simplify the training of the regression model.

In step 405, a multiple regression fit is performed on the subset of parameters and the outcome. For example, a binomial logistic classification model is fit with backward elimination to produce a refined subset of training data parameters and to produce an output probability of a POUR outcome, e.g., using a fitting procedure as illustrated in FIG. 2. Some parameters initially included might be eliminated by backward elimination based on significant changes in likelihood ratios, using a 0.10 cutoff. If eliminating the parameter changed the likelihood ratio by less than 0.1, the parameter was eliminated. The result is a set of coefficients for the revised subset of parameters, which cause the errors, when run on the training set data, to cause the minimal change in the model likelihood ratios.

In step 407, the regression model is used in a binomial logistic classification model by selecting a cutoff that gives good performance in terms of true positive rates and true negative rates. FIG. 5A is a plot that illustrates example classification performance based on a cutoff applied to model output, according to an embodiment. The development of the target outcome, such as POUR, is viewed as a stochastic process in which even though all known inputs are the same the result can be different, but the deviations follow certain probabilities represented by probability density functions. In FIG. 5 the horizontal axis 502 indicates model output X. For a given model output the probability of the subject being in a first class Y=0 (e.g., the non-POUR outcome class) is given by trace 507 and the probability of being in a second class Y=1 (e.g., the POUR outcome class) is given by trace 508. The total probability of a certain model result x is given by the sum of the two curves given by trace 506. In binomial classification, model results below a cutoff 506 are considered to be in class Y=0, while model results above the cutoff 506 are considered to be in the other class Y=1. For any such cutoff there will be a certain probability of true negative result given by the area labeled TN, a false negative result given by the area FN, a true positive result given by the area TP, and a false positive result given by the area FP. By changing the cutoff all these areas and the corresponding rates change. Using the available data, the cutoff is varied until the TN and TP results, or some other measure, are acceptable for some practical application.

A good measure often used in balancing TP and TN rates with FP and FN rates are the receiver operating characteristic curve, or ROC curve, also sometimes called a relative operating characteristic curve. A ROC curve is a graphical plot that illustrates the diagnostic ability of a binary classifier system as its discrimination threshold is varied. The method was originally developed for operators of military radar receivers starting in 1941, which led to its name. The ROC curve is created by plotting the true positive rate (TPR) against the false positive rate (FPR) at various threshold settings (e.g., cutoffs). The true-positive rate is also known as sensitivity, recall or probability of detection. The false-positive rate is also known as probability of false alarm and can be calculated as (1−specificity). When the performance is calculated from just a sample of the population, it can be thought of as estimators of these quantities. In general, if the probability distributions for both detection and false alarm are known, the ROC curve can be generated by plotting the cumulative distribution function (area under the probability distribution as depicted in FIG. 5A.

Selecting a cutoff that provides a favorable point on the ROC curve is what occurs in step 407 for the output of the multiple regression model. In some embodiments step 407 is delayed until after there is also a neural network model and the cutoffs for both model outputs are determined in concert in step 415, described below.

In step 411, the values of every training data parameter is scaled (e.g., by an additive or multiplicative factor or both) so that all the values for that parameter in the training set lie in a limited range, such as 0 to 1 inclusive, or −1 to 1 inclusive. In such a scaling, parameters that are either yes or no (e.g., the subject has diabetes or not) can be represented by the extremes (either 1 and 0, or 1 and −1) and still compete for significant weights during training of the neural network with parameters that have larger ranges, e.g., patient temperatures near 100 degrees Fahrenheit.

In step 413 a neural network is trained on the training set data scaled parameter values. In various embodiments, the input layer of the neural network has a node for each parameter in the training set and an output layer that has one node that expresses the probability of the outcome of interest, e.g., the probability of a POUR outcome. For example, during training a retrospective subject who develops POUR has a 1 placed in the output node and a retrospective subject who does not develop POUR has a 0 placed in the output node. Any neural network may be used; but, having too many layers with too many nodes taxes the solution and demands larger training sets. In the example embodiment presented in the Appendix it was found that a neural network with two hidden layers fully connected to the preceding layers with 38 nodes and 21 nodes, respectively, a sigmoid activation function at each node, and an identity output activation function, performed well. It is expected that other neural networks with a similar number of hidden layers and nodes per hidden layer would also perform well. For example, a range of two to three hidden layers each with 10 to 80 nodes and use of different activation functions are expected to perform as well as the neural network described herein. A practitioner can determine a number of nodes in each hidden layer by starting with an initial guess for the number of nodes (e.g., based on a number larger than, e.g., twice as large as, the number of parameters expected to be important a priori) in the first layer and something on the order of half that number in the second hidden layer, and again half in each successive layer. The number of nodes in each layer can then be adjusted up or down to see the effect on the performance of the model and stopping when the performance seems to be sufficient for an intended purpose, e.g. to distinguish between those persons to be treated for the outcome and those not treated or treated differently.

In step 415, the classification cutoff for the output of the neural network is determined, e.g., using an approach as described above in step 407 for the multiple regression model. In some embodiments, all combinations of cutoff points to the nearest 0.01 (1% chance) for each model were used, and the statistical outcomes for that combination of cutoff points was tested. According to a strict test, the prediction estimate from each model exceeds its respective cutoff point to designate the patient as predicted to get the particular outcome (e.g., POUR). According to a loose test, the prediction estimate from either model exceeds its respective cutoff point to designate the patient as predicted to get the particular outcome (e.g., POUR).

By the end of step 415, a processor is configured to process a new subject (called a patient or current subject in the following) whose outcome is not yet known. The treatment of that patient is based at least in part on predicting the outcome for that patient using the values of the input parameters for that patient and the models configured above. For example, catheterization or medicament administration or some combination for that patient is prescribed based at least in part on predicting a POUR outcome using the values of the input parameters for that patient and the models configured for POUR outcomes

In step 421, patient and procedure information is obtained for a current subject with unknown outcome. Any method may be used to obtain this information, such as described above for the training set data in step 401. For example, values for 250 parameters describing patient and lower spine surgery information are obtained for a patient before or during surgery, before there is a POUR outcome.

In step 423, the regression model or neural network or both are operated using as input all or a revised subset, respectively, of the information obtained in step 421 for the current subject. In step 426 the strict or loose test is applied to determine whether the current subject is predicted to have the outcome of interest, e.g., POUR. Step 426 includes sending any signal to a human or automated caregiver to indicate the outcome classification for the current subject. For example, a signal that indicates a POUR classification (yes or no) for the subject is sent based at least in part on the neural network output data. In some embodiments, the POUR classification for the subject is further based at least in part on the binomial regression output data.

If the signal indicates a yes classification for the outcome, control passes to step 431 to treat the current subject based on predicting the outcome of interest. For example, when POUR is predicted, a POUR intervention therapy can be administered to the patient. A “POUR intervention therapy” refers to administration of an agent and/or application of a clinical procedure to a patient determined to be a risk of POUR, and is one that reduces or ameliorates the severity, duration, or progression of the disorder being treated (e.g., POUR), prevent the advancement of the disorder being treated (e.g., POUR), cause the regression of the disorder being treated (e.g., POUR. Examples of a POUR intervention therapy include intraoperative bladder catheter placement, immediate postoperative bladder catheterization if bladder volume is >450 mL, administration of opioid-sparing postoperative analgesia (e.g., gabapentin), and/or administration of a detrusor relaxant, such as alpha-1 antagonists.

If the signal indicates a no classification for the outcome, control passes to step 433 to treat the current subject as not having the outcome of interest. For example, when no POUR is predicted the patient is given different or enhanced anesthesia during the remainder of the surgery, or is not given medication to mediate POUR, or is not catheterized during or after surgery. After either step 431 or step 433, control passes to step 435.

In step 435, the actual outcome for the patient is observed. In some embodiments, after the actual outcome is observed, the information for the patient and the patient's outcome is added to a validation or training set data structures for updating the models or the cutoffs for the models or both.

In step 441, it is determined whether the models or cutoffs should be updates. If so, control passes back to step 405 and following to again fit the models or cutoffs to the new updated training or validation data. If not, control passes to step 443.

In step 443, it is determined whether there is another new subject (patient) for whom to predict the outcome and treatment. If so, control passes back to step 421 and following to apply the classification models to the new patient. If not, the process ends.

FIG. 5B is a flow chart that illustrates an example method to stack two machine learning models to classify a particular subject, according to an embodiment of step 415 of the method of FIG. 4. As stated above, each model outputs a prediction of the patient experiencing the outcome of interest from 0 to 1, whereas 1 represents a 100% chance. In the method of FIG. 5B, one filters through all combinations of cutoff points to the nearest 0.01 (1% chance) for each model, and to test the statistical outcomes for that combination of cutoff points. This is done in 2 ways: 1) a strict test such that the prediction estimate needed to each both respective cutoff points to designate the patient as predicted to get POUR need to be met and 2) a loose test such that only one cutoff point needed to be exceeded. For each combination of cutoff points, and for each type of test (strict and loose), a testing or validation set can be used to measure the predictive outcomes of combining the models: sensitivity; specificity; negative predictive power (NPV); positive predictive power (PPV); average sensitivity and PPV; average specificity and NPV; average sensitivity and specificity; average NPV and PPV; average sensitivity, specificity, NPV, and PPV; and accuracy. In the strict test, for example, if the logistic regression model was greater than the logistic regression cutoff and the neural network model was greater than the neural network cutoff, the final prediction was that the patient would develop the outcome. If instead, the logistic regression model was less than the logistic regression cutoff or the neural network model was less than the neural network cutoff, the final prediction was that the patient would not develop the outcome. This is different in other stacking methods as this is not regression or ensemble; but is, rather, a logical ensemble.

FIG. 5C is a plot of relative operating character (ROC) curves that illustrates an example performance when stacking two machine learning models to classify a particular subject, according to an embodiment. Each point along the curve represents a different cutoff point for the model. In a ROC curve, a model with no skill is represented by the diagonal line. No matter the cutoff, both the true positive rate and the false positive rate increase together. The best skill is represented by a point furthest from this line, provided by the cutoff value associated with that point. Traces on this plot show the performance for the regression model (short dashed trace), the neural network (wide spaced dashed trace) and combined using the strict test (soldi trace). Code was written to test combinations of each point along these curves to find the optimal combination of cutoff points that maximizes the prediction of a combination of the models. The strict test selects follows the best performance of the two models.

### 3. EXAMPLE EMBODIMENTS

in more detail in the Appendix. Statements made in the Appendix apply only to the embodiments in the Appendix.

### 4. COMPUTATIONAL HARDWARE OVERVIEW

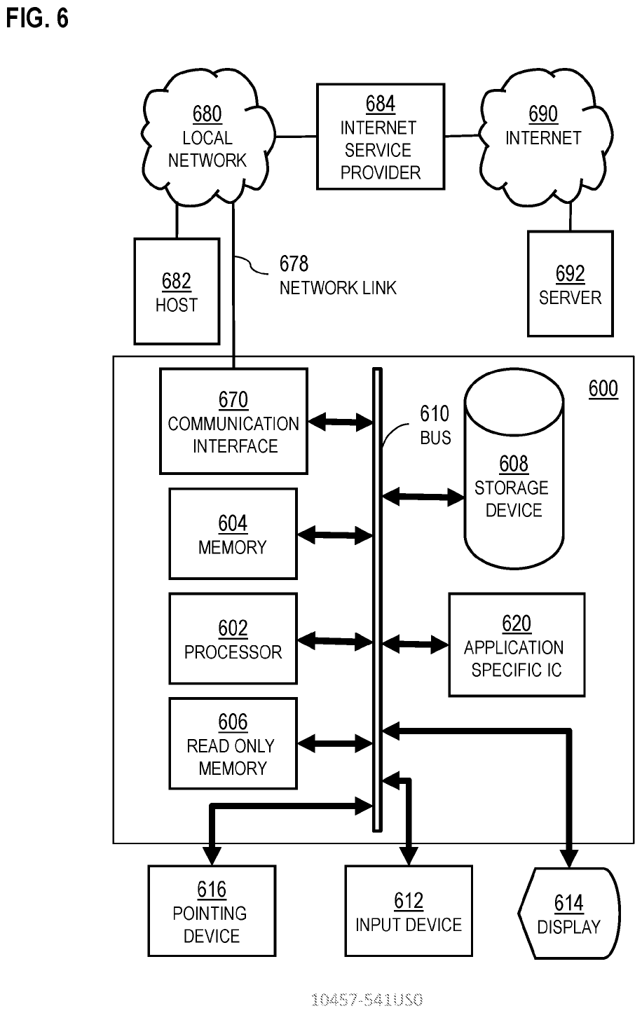

FIG. 6 is a block diagram that illustrates a computer system 600 upon which an embodiment of the invention may be implemented. Computer system 600 includes a communication mechanism such as a bus 610 for passing information between other internal and external components of the computer system 600. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 600, or a portion thereof, constitutes a means for performing one or more steps of one or more methods described herein.

A sequence of binary digits constitutes digital data that is used to represent a number or code for a character. A bus 610 includes many parallel conductors of information so that information is transferred quickly among devices coupled to the bus 610. One or more processors 602 for processing information are coupled with the bus 610. A processor 602 performs a set of operations on information. The set of operations include bringing information in from the bus 610 and placing information on the bus 610. The set of operations also typically include comparing two or more units of information, shifting positions of units of information, and combining two or more units of information, such as by addition or multiplication. A sequence of operations to be executed by the processor 602 constitutes computer instructions.

Computer system 600 also includes a memory 604 coupled to bus 610. The memory 604, such as a random access memory (RAM) or other dynamic storage device, stores information including computer instructions. Dynamic memory allows information stored therein to be changed by the computer system 600. RAM allows a unit of information stored at a location called a memory address to be stored and retrieved independently of information at neighboring addresses. The memory 604 is also used by the processor 602 to store temporary values during execution of computer instructions. The computer system 600 also includes a read only memory (ROM) 606 or other static storage device coupled to the bus 610 for storing static information, including instructions, that is not changed by the computer system 600. Also coupled to bus 610 is a non-volatile (persistent) storage device 608, such as a magnetic disk or optical disk, for storing information, including instructions, that persists even when the computer system 600 is turned off or otherwise loses power.

Information, including instructions, is provided to the bus 610 for use by the processor from an external input device 612, such as a keyboard containing alphanumeric keys operated by a human user, or a sensor. A sensor detects conditions in its vicinity and transforms those detections into signals compatible with the signals used to represent information in computer system 600. Other external devices coupled to bus 610, used primarily for interacting with humans, include a display device 614, such as a cathode ray tube (CRT) or a liquid crystal display (LCD), for presenting images, and a pointing device 616, such as a mouse or a trackball or cursor direction keys, for controlling a position of a small cursor image presented on the display 614 and issuing commands associated with graphical elements presented on the display 614.

In the illustrated embodiment, special purpose hardware, such as an application specific integrated circuit (IC) 620, is coupled to bus 610. The special purpose hardware is configured to perform operations not performed by processor 602 quickly enough for special purposes. Examples of application specific ICs include graphics accelerator cards for generating images for display 614, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 600 also includes one or more instances of a communications interface 670 coupled to bus 610. Communication interface 670 provides a two-way communication coupling to a variety of external devices that operate with their own processors, such as printers, scanners and external disks. In general the coupling is with a network link 678 that is connected to a local network 680 to which a variety of external devices with their own processors are connected. For example, communication interface 670 may be a parallel port or a serial port or a universal serial bus (USB) port on a personal computer. In some embodiments, communications interface 670 is an integrated services digital network (ISDN) card or a digital subscriber line (DSL) card or a telephone modem that provides an information communication connection to a corresponding type of telephone line. In some embodiments, a communication interface 670 is a cable modem that converts signals on bus 610 into signals for a communication connection over a coaxial cable or into optical signals for a communication connection over a fiber optic cable. As another example, communications interface 670 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 670 sends and receives electrical, acoustic or electromagnetic signals, including infrared and optical signals, that carry information streams, such as digital data.

The term computer-readable medium is used herein to refer to any medium that participates in providing information to processor 602, including instructions for execution. Such a medium may take many forms, including, but not limited to, non-volatile media, volatile media and transmission media. Non-volatile media include, for example, optical or magnetic disks, such as storage device 608. Volatile media include, for example, dynamic memory 604. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 602, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 602, except for carrier waves and other signals.

Logic encoded in one or more tangible media includes one or both of processor instructions on a computer-readable storage media and special purpose hardware, such as ASIC 620.

Network link 678 typically provides information communication through one or more networks to other devices that use or process the information. For example, network link 678 may provide a connection through local network 680 to a host computer 682 or to equipment 684 operated by an Internet Service Provider (ISP). ISP equipment 684 in turn provides data communication services through the public, world-wide packet-switching communication network of networks now commonly referred to as the Internet 690. A computer called a server 692 connected to the Internet provides a service in response to information received over the Internet. For example, server 692 provides information representing video data for presentation at display 614.

The invention is related to the use of computer system 600 for implementing the techniques described herein. According to one embodiment of the invention, those techniques are performed by computer system 600 in response to processor 602 executing one or more sequences of one or more instructions contained in memory 604. Such instructions, also called software and program code, may be read into memory 604 from another computer-readable medium such as storage device 608. Execution of the sequences of instructions contained in memory 604 causes processor 602 to perform the method steps described herein. In alternative embodiments, hardware, such as application specific integrated circuit 620, may be used in place of or in combination with software to implement the invention. Thus, embodiments of the invention are not limited to any specific combination of hardware and software.

The signals transmitted over network link 678 and other networks through communications interface 670, carry information to and from computer system 600. Computer system 600 can send and receive information, including program code, through the networks 680, 690 among others, through network link 678 and communications interface 670. In an example using the Internet 690, a server 692 transmits program code for a particular application, requested by a message sent from computer 600, through Internet 690, ISP equipment 684, local network 680 and communications interface 670. The received code may be executed by processor 602 as it is received, or may be stored in storage device 608 or other non-volatile storage for later execution, or both. In this manner, computer system 600 may obtain application program code in the form of a signal on a carrier wave.

Various forms of computer readable media may be involved in carrying one or more sequence of instructions or data or both to processor 602 for execution. For example, instructions and data may initially be carried on a magnetic disk of a remote computer such as host 682. The remote computer loads the instructions and data into its dynamic memory and sends the instructions and data over a telephone line using a modem. A modem local to the computer system 600 receives the instructions and data on a telephone line and uses an infra-red transmitter to convert the instructions and data to a signal on an infra-red a carrier wave serving as the network link 678. An infrared detector serving as communications interface 670 receives the instructions and data carried in the infrared signal and places information representing the instructions and data onto bus 610. Bus 610 carries the information to memory 604 from which processor 602 retrieves and executes the instructions using some of the data sent with the instructions. The instructions and data received in memory 604 may optionally be stored on storage device 608, either before or after execution by the processor 602.

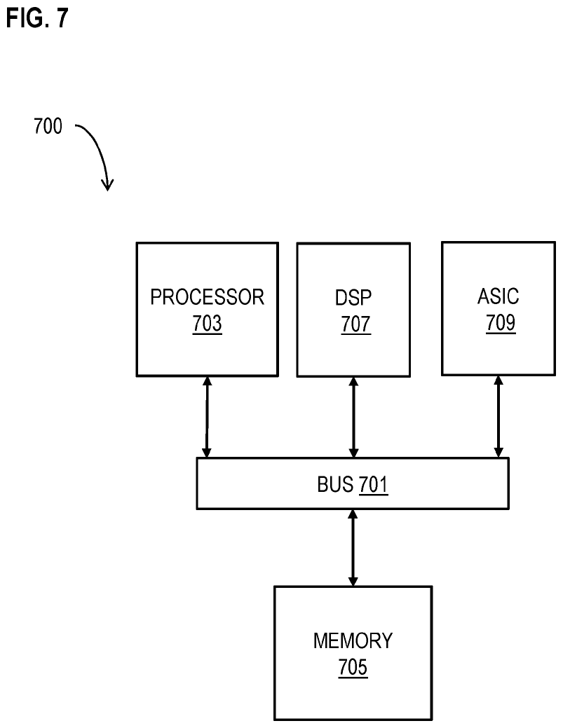

FIG. 7 illustrates a chip set 700 upon which an embodiment of the invention may be implemented. Chip set 700 is programmed to perform one or more steps of a method described herein and includes, for instance, the processor and memory components described with respect to FIG. 6 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 700, or a portion thereof, constitutes a means for performing one or more steps of a method described herein.

In one embodiment, the chip set 700 includes a communication mechanism such as a bus 701 for passing information among the components of the chip set 700. A processor 703 has connectivity to the bus 701 to execute instructions and process information stored in, for example, a memory 705. The processor 703 may include one or more processing cores with each core configured to perform independently. A multi-core processor enables multiprocessing within a single physical package. Examples of a multi-core processor include two, four, eight, or greater numbers of processing cores. Alternatively or in addition, the processor 703 may include one or more microprocessors configured in tandem via the bus 701 to enable independent execution of instructions, pipelining, and multithreading. The processor 703 may also be accompanied with one or more specialized components to perform certain processing functions and tasks such as one or more digital signal processors (DSP) 707, or one or more application-specific integrated circuits (ASIC) 709. A DSP 707 typically is configured to process real-world signals (e.g., sound) in real time independently of the processor 703. Similarly, an ASIC 709 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 703 and accompanying components have connectivity to the memory 705 via the bus 701. The memory 705 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 705 also stores the data associated with or generated by the execution of one or more steps of the methods described herein.

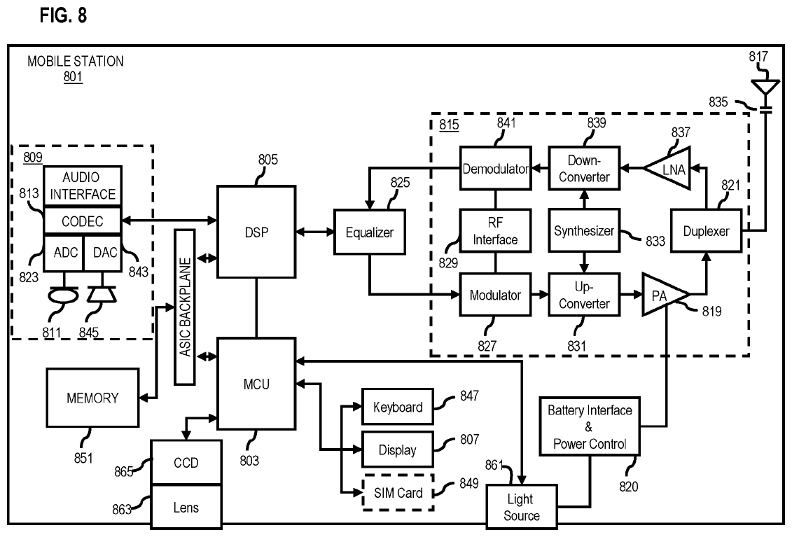

FIG. 8 is a diagram of exemplary components of a mobile terminal 800 (e.g., cell phone handset) for communications, which is capable of operating in the system of FIG. 2B, according to one embodiment. In some embodiments, mobile terminal 801, or a portion thereof, constitutes a means for performing one or more steps described herein. Generally, a radio receiver is often defined in terms of front-end and back-end characteristics. The front-end of the receiver encompasses all of the Radio Frequency (RF) circuitry whereas the back-end encompasses all of the base-band processing circuitry. As used in this application, the term “circuitry” refers to both: (1) hardware-only implementations (such as implementations in only analog and/or digital circuitry), and (2) to combinations of circuitry and software (and/or firmware) (such as, if applicable to the particular context, to a combination of processor(s), including digital signal processor(s), software, and memory (ies) that work together to cause an apparatus, such as a mobile phone or server, to perform various functions). This definition of “circuitry” applies to all uses of this term in this application, including in any claims. As a further example, as used in this application and if applicable to the particular context, the term “circuitry” would also cover an implementation of merely a processor (or multiple processors) and its (or their) accompanying software/or firmware. The term “circuitry” would also cover if applicable to the particular context, for example, a baseband integrated circuit or applications processor integrated circuit in a mobile phone or a similar integrated circuit in a cellular network device or other network devices.

Pertinent internal components of the telephone include a Main Control Unit (MCU) 803, a Digital Signal Processor (DSP) 805, and a receiver/transmitter unit including a microphone gain control unit and a speaker gain control unit. A main display unit 807 provides a display to the user in support of various applications and mobile terminal functions that perform or support the steps as described herein. The display 807 includes display circuitry configured to display at least a portion of a user interface of the mobile terminal (e.g., mobile telephone). Additionally, the display 807 and display circuitry are configured to facilitate user control of at least some functions of the mobile terminal. An audio function circuitry 809 includes a microphone 811 and microphone amplifier that amplifies the speech signal output from the microphone 811. The amplified speech signal output from the microphone 811 is fed to a coder/decoder (CODEC) 813.

A radio section 815 amplifies power and converts frequency in order to communicate with a base station, which is included in a mobile communication system, via antenna 817. The power amplifier (PA) 819 and the transmitter/modulation circuitry are operationally responsive to the MCU 803, with an output from the PA 819 coupled to the duplexer 821 or circulator or antenna switch, as known in the art. The PA 819 also couples to a battery interface and power control unit 820.

In use, a user of mobile terminal 801 speaks into the microphone 811 and his or her voice along with any detected background noise is converted into an analog voltage. The analog voltage is then converted into a digital signal through the Analog to Digital Converter (ADC) 823. The control unit 803 routes the digital signal into the DSP 805 for processing therein, such as speech encoding, channel encoding, encrypting, and interleaving. In one embodiment, the processed voice signals are encoded, by units not separately shown, using a cellular transmission protocol such as enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium. e.g., microwave access (WiMAX), Long Term Evolution (LTE) networks, code division multiple access (CDMA), wideband code division multiple access (WCDMA), wireless fidelity (WiFi), satellite, and the like, or any combination thereof.

The encoded signals are then routed to an equalizer 825 for compensation of any frequency-dependent impairments that occur during transmission though the air such as phase and amplitude distortion. After equalizing the bit stream, the modulator 827 combines the signal with a RF signal generated in the RF interface 829. The modulator 827 generates a sine wave by way of frequency or phase modulation. In order to prepare the signal for transmission, an up-converter 831 combines the sine wave output from the modulator 827 with another sine wave generated by a synthesizer 833 to achieve the desired frequency of transmission. The signal is then sent through a PA 819 to increase the signal to an appropriate power level. In practical systems, the PA 819 acts as a variable gain amplifier whose gain is controlled by the DSP 805 from information received from a network base station. The signal is then filtered within the duplexer 821 and optionally sent to an antenna coupler 835 to match impedances to provide maximum power transfer. Finally, the signal is transmitted via antenna 817 to a local base station. An automatic gain control (AGC) can be supplied to control the gain of the final stages of the receiver. The signals may be forwarded from there to a remote telephone which may be another cellular telephone, any other mobile phone or a land-line connected to a Public Switched Telephone Network (PSTN), or other telephony networks.

Voice signals transmitted to the mobile terminal 801 are received via antenna 817 and immediately amplified by a low noise amplifier (LNA) 837. A down-converter 839 lowers the carrier frequency while the demodulator 841 strips away the RF leaving only a digital bit stream. The signal then goes through the equalizer 825 and is processed by the DSP 805. A Digital to Analog Converter (DAC) 843 converts the signal and the resulting output is transmitted to the user through the speaker 845, all under control of a Main Control Unit (MCU) 803 which can be implemented as a Central Processing Unit (CPU) (not shown).

The MCU 803 receives various signals including input signals from the keyboard 847. The keyboard 847 and/or the MCU 803 in combination with other user input components (e.g., the microphone 811) comprise a user interface circuitry for managing user input. The MCU 803 runs a user interface software to facilitate user control of at least some functions of the mobile terminal 801 as described herein. The MCU 803 also delivers a display command and a switch command to the display 807 and to the speech output switching controller, respectively. Further, the MCU 803 exchanges information with the DSP 805 and can access an optionally incorporated SIM card 849 and a memory 851. In addition, the MCU 803 executes various control functions required of the terminal. The DSP 805 may, depending upon the implementation, perform any of a variety of conventional digital processing functions on the voice signals. Additionally, DSP 805 determines the background noise level of the local environment from the signals detected by microphone 811 and sets the gain of microphone 811 to a level selected to compensate for the natural tendency of the user of the mobile terminal 801.

The CODEC 813 includes the ADC 823 and DAC 843. The memory 851 stores various data including call incoming tone data and is capable of storing other data including music data received via, e.g., the global Internet. The software module could reside in RAM memory, flash memory, registers, or any other form of writable storage medium known in the art. The memory device 851 may be, but not limited to, a single memory, CD, DVD, ROM, RAM, EEPROM, optical storage, magnetic disk storage, flash memory storage, or any other non-volatile storage medium capable of storing digital data.

An optionally incorporated SIM card 849 carries, for instance, important information, such as the cellular phone number, the carrier supplying service, subscription details, and security information. The SIM card 849 serves primarily to identify the mobile terminal 801 on a radio network. The card 849 also contains a memory for storing a personal telephone number registry, text messages, and user specific mobile terminal settings.

In some embodiments, the mobile terminal 801 includes a digital camera comprising an array of optical detectors, such as charge coupled device (CCD) array 865. The output of the array is image data that is transferred to the MCU for further processing or storage in the memory 851 or both. In the illustrated embodiment, the light impinges on the optical array through a lens 863, such as a pin-hole lens or a material lens made of an optical grade glass or plastic material. In the illustrated embodiment, the mobile terminal 801 includes a light source 861, such as a LED to illuminate a subject for capture by the optical array, e.g., CCD 865. The light source is powered by the battery interface and power control module 820 and controlled by the MCU 803 based on instructions stored or loaded into the MCU 803.

### 4: EXAMPLES

### Preoperative Prediction of Postoperative Urinary Retention in Lumbar Surgery: A Comparison of Regression to Multilayer Neural Network

An example embodiment using a particular neural network and multiple regression model in concert with corresponding cutoffs for POUR outcomes of lower spinal surgeries is described below:

### Methods

### Part 1: Study Design and Setting

This was a retrospective review approved by the local IRB and was aimed at developing an optimal model to predict POUR. The first part comprised a retrospective review of consecutive adult patients who underwent lumbar spine surgery between Jun. 1, 2017, and Jun. 1, 2019, at the University of Florida. Patients were excluded if they required emergency surgery, were <18 years old, or had surgery in a nonlumbar region (thoracic or cervical). The second part comprised development of two machine learning techniques: a binomial logistic regression and an artificial neural network classification. These models were furthermore combined to optimize prediction strength.

### Case Ascertainment

POUR was defined according to previous literature as reinsertion of a Foley catheter based on retention urine volume>400 mL, or requiring straight catheterization for urine volumes>400 mL.^{4,22-27} Urine volume was determined per standard of care with nurse-led bladder scanning.

### Clinical Variables

The patient characteristics, including all preexisting ICD-10 codes associated with the patient; age; sex; body mass index (BMI); preoperative opioid use (morphine, methadone, fentanyl, oxycodone, hydrocodone, meperidine); preoperative urinary retention medication use (tamsulosin, doxazosin); planned surgery specifics; and POUR, were collected and assessed. Hospital LOS was also recorded.

### Part 2: Statistical Analysis

### Identification of Predictors

Univariate tests were used on the entire set to discover factors to include in multivariate analyses, and a Bonferroni correction was used to correct for multiple analyses. Mann-Whitney U-tests were used for continuous and nominal variables, whereas chi-square tests were used for categorical variables. Kruskal-Wallis tests were used to compare training, validation, and testing sets. Hospital LOS was not included in the final models because this was an outcome measure.

### Model Derivation

The data were split into training, validation, and testing sets using an approximately 65:10:25 ratio.^{28,29} A binomial logistic model—which estimates the probability that an outcome is present given the values of explanatory variables and is typically used for classification—was formed with backward elimination based on significant changes in likelihood ratios, using a 0.10 cutoff.^{30,31} All patient demographics and surgical characteristics were included in the model, but only comorbidities that had significant correlations with POUR were included (p<0.05 corrected for multiple comparisons).

We used a multilayer perceptron (MLP) neural network architecture-attractive because it demonstrates an ability to learn salient features of the data on its own-which consisted of two hidden layers terminating at an output layer.¹⁹ The first hidden layer consisted of 38 fully connected nodes, whereas the second layer consisted of 21 fully connected nodes. Hidden layers used a sigmoid activation function with no dropout. The output layer used an identity activation function and a sum of squares error function. The stopping rule was 1 consecutive step with no decrease in error based on the validation set.

Three additional regression models were developed from previously published models adjusted to include only relevant factors, to derive a pragmatic preoperative risk assessment tool (i.e., factors known preoperatively and applied to the lumbar spine).

### Model Validation

Using the same training/validation/testing split on both the regression and neural network models, optimal models were selected by maximizing both adjusted R² and validation set accuracy. Performance of the chosen model was then evaluated on the testing set. Performance was measured on validation and testing sets combined because there was no need for a validation step.

### Model Stacking

The models were combined such that if a threshold cutoff point (different for each model) was exceeded by either model, the classification was declared to be positive (i.e., the patient is predicted to develop POUR).³² Outcomes from all cutoff points from 0.01 to 0.99 for each model were compared to maximize each individual outcome measure and combinations of outcome measures (i.e., average sensitivity and positive predictive value [PPV], average specificity and negative predictive value [NPV], average sensitivity and specificity, average NPV and PPV, and the average of all outcome measures).

All statistical analyses were performed with SPSS version 23 (IBM Corp.). Cutoff points were discovered using code developed with Strawberry Perl 5.30.2.1.

### Results

### Clinical Characteristics

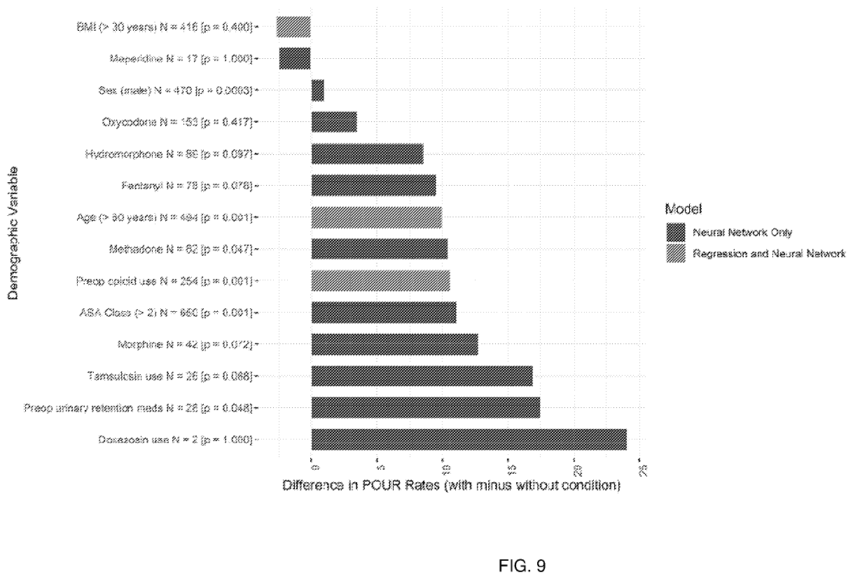

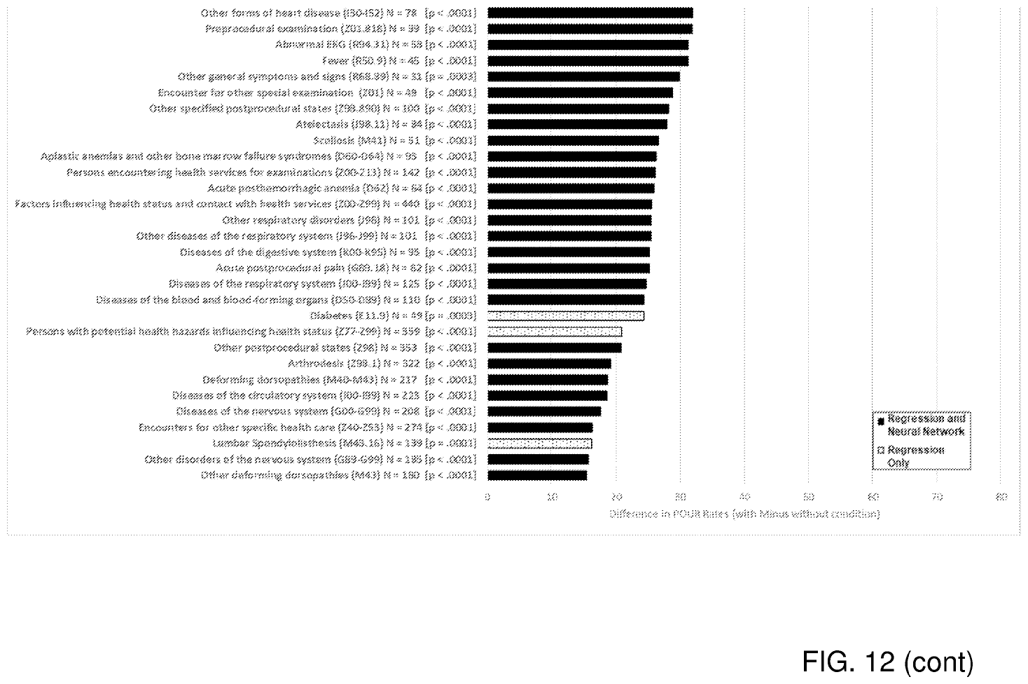

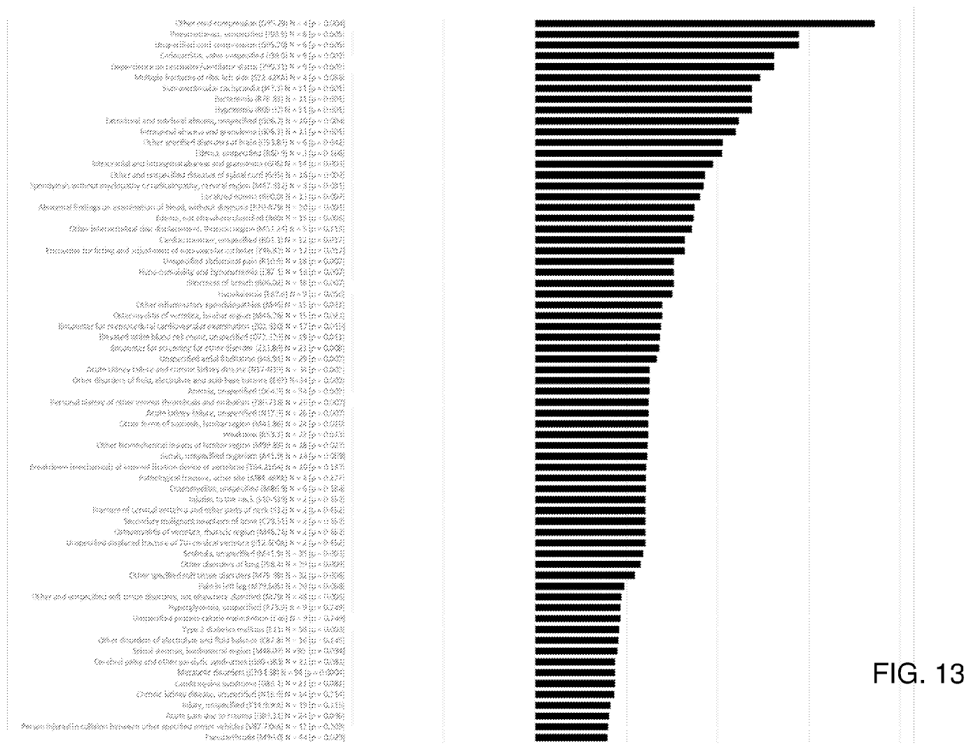

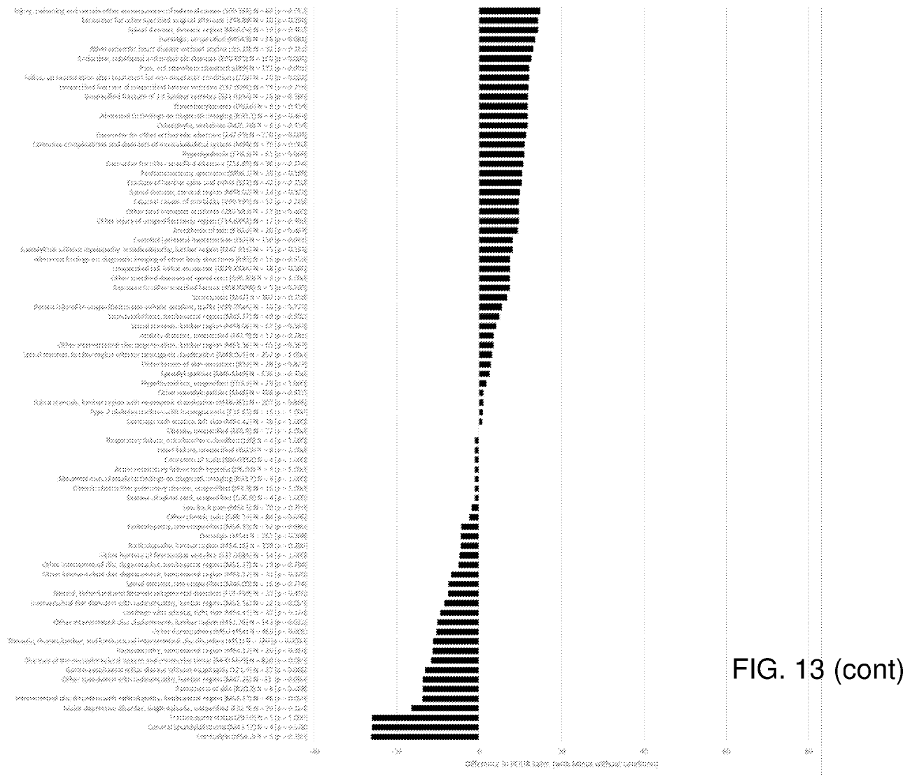

There were a total of 1311 patients who underwent elective spine surgery in the study period. Of those, 891 were included in the analysis, with 369 excluded because they were cervical or thoracic surgeries and 51 excluded due to missing data. POUR rates were found to be 25.9% in the entire cohort. Differences in patient demographics among the training/validation/testing split are shown in Table 2. The mean age was 59.6±15.5 years, 52.7% were male, the mean BMI was 30.4±6.4, the mean American Society of Anesthesiologists (ASA) class was 2.8±0.6, and there was a mean of 5.6±5.7 comorbidities. The training, validation, and testing sets did not differ significantly among age, sex, or BMI. The validation set had significantly higher ASA class and fewer comorbidities. Conversely, the training and testing sets did not vary significantly between these factors. Patient demographics and their designated univariate analyses are demonstrated in FIG. 9. Patients who developed POUR were significantly older than those who did not (62.5±15.1 years vs 58.6±15.5 years; p=0.0003) and were more likely to use preoperative opioids (36.8% vs 25.6%; p=0.001) or preoperative urinary retention medications (5.2% vs 2.4%; p=0.048). Male sex (53.7% vs 52.4%; p=0.760) and BMI (30.2±5.9 vs 30.5±6.6; p=0.682) were not found to be significantly different between groups. Additionally, rates of POUR were found to be significantly higher in cases associated with 65 separate preoperative ICD-10 codes or groups of ICD-10 codes, including history of urinary retention or UTIs. The differences in POUR rates for these traits are demonstrated in FIG. 12. The differences in POUR rates for all nonsignificant ICD codes are demonstrated in FIG. 13. Hospital LOS for patients with POUR was significantly longer than for those without POUR (6.9±9.5 days vs 2.7±3.0 days; p<0.0001).

TABLE 2
Patient Demographics of Overall and Train-Validation-Test Splits

| | | Training | Validation | Testing | |
|---|---|---|---|---|---|
| | Overall | Set | Set | Set | p-value |
| Frequency | 231 | 150 | 22 | 59 | — |
| POUR Rate (%) | 25.9 | 26.7 | 23.4 | 25.1 | 0.754 |
| Age (Mean ± SD) | 59.6 ± 15.5 | 58.7 ± 15.5 | 57.6 ± 15.1 | 60.2 ± 15.7 | 0.282 |
| Male Sex (%) | 52.7 | 55.3 | 45.7 | 49.4 | 0.109 |
| BMI (Mean ± SD) | 30.4 ± 6.4 | 30.3 ± 6.3 | 30.7 ± 7.5 | 30.7 ± 6.2 | 0.684 |
| ASA Class (Mean ± SD) | 2.8 ± 0.6 | 2.7 ± 0.5 | 3.7 ± 0.6 | 2.7 ± 0.6 | <0.001 |
| Number of Comorbidities | 5.6 ± 5.7 | 6.1 ± 6.1 | 2.7 ± 4.1 | 5.7 ± 4.9 | <0.001 |
| (Mean ± SD) | | | | | |

### Surgical Characteristics

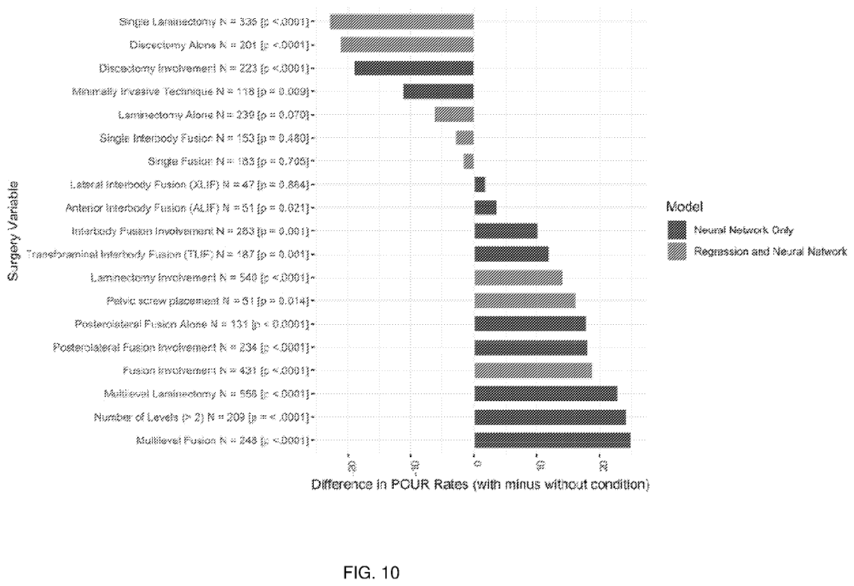

The difference in POUR rates for individual surgical characteristics, as demonstrated in FIG. 10, revealed multiple surgical predictors of POUR. Rates of POUR were significantly lower in discectomies compared to patients whose spine surgery did not include discectomies (11.7% vs 30.7%; p<0.0001) even as part of the operation. Rates of POUR were found to be higher overall in patients getting laminectomies (31.5% vs 17.4%; p<0.0001) but not when only a laminectomy was performed (21.3% vs 27.6%; p=0.070). Furthermore, rates were found to be significantly higher in multilevel laminectomies (34.5% vs 11.6%; p<0.0001) and significantly lower in single-level laminectomies (11.6% vs 34.5%; p<0.0001). Similarly, rates of POUR were found to be higher in surgeries with a fusion component (35.7% vs 16.7%; p<0.0001), except for single-level fusions (24.6% vs 26.3%; p=0.705). Within the scope of fusion surgeries, posterolateral fusions showed significantly higher rates of POUR (involvement: 39.3% vs 21.2%; alone: 41.2% vs 23.3%; p<0.0001 for both), as did interbody fusions (32.9% vs 22.7%; p=0.001) and pelvic screw placement (41.2% vs 25.0%; p=0.014). Minimally invasive techniques demonstrated a significantly lower rate of POUR (16.1% vs 27.4%; p=0.009). The average number of vertebral levels operated on was found to be 1.8±1.8 in those who did not develop POUR and 2.9±2.8 in those who did (p<0.0001).

### Outcomes

Binomial logistic multivariate model results are demonstrated in Table 3. Of the factors included in the model, only ICD-10 codes for diabetes (E11.9), abnormal heartbeat (R00), other general symptoms and signs (R68.89), altered mental status (R41.82), and screening for cardiovascular disorders (Z13.6) in addition to plans for a single laminectomy were found to be significant predictors of change in POUR. The ICD code for “other general symptoms and signs” and plans for only a single-level laminectomy were found to be significantly protective against POUR. For brevity, specific results of the neural network model were not included.

TABLE 3
Multivariate Binomial Logistic Regression Analysis

| | | | | Odds | | |
|---|---|---|---|---|---|---|
| | | | p- | Ratio | 95% CI for OR | |
| | Effect | SE | value | (OR) | Lower | Upper |
| Age (years) | .002 | .008 | .780 | 1.002 | .986 | 1.019 |
| Pre-operative opioid use | −.121 | .273 | .658 | .886 | .520 | 1.512 |
| Body Mass Index (kg/m2) | −.006 | .019 | .759 | .994 | .958 | 1.032 |
| Diabetes (E11.9) | .953 | .485 | .050 | 2.593 | 1.001 | 6.713 |
| Cardiomegaly (I51.7) | 1.070 | .571 | .061 | 2.916 | .952 | 8.933 |
| Hypotension (I95.9) | .842 | .535 | .115 | 2.322 | .813 | 6.629 |
| Ileus (K56.7) | 1.240 | .844 | .142 | 3.455 | .660 | 18.077 |
| Constipation (K59.00) | 1.192 | .657 | .070 | 3.293 | .909 | 11.926 |
| Other Intestinal Disease (K63.89) | .725 | .653 | .267 | 2.064 | .574 | 7.420 |
| Spondylolisthesis (M43.16) | .408 | .312 | .191 | 1.504 | .815 | 2.772 |
| UTI (N39.0) | 1.405 | 1.001 | .161 | 4.075 | .573 | 28.996 |
| Abnormalities of heart beat (R00) | .982 | .446 | .028 | 2.670 | 1.114 | 6.395 |
| Other general symptoms and signs | −1.605 | .705 | .023 | .201 | .050 | .800 |
| (R68.89) | | | | | | |
| Altered Mental Status (R41.82) | 3.013 | 1.186 | .011 | 20.356 | 1.990 | 208.226 |
| Urinary Retention (R33.9) | 1.103 | .759 | .146 | 3.013 | .681 | 13.330 |
| Pain (R52) | 1.204 | .729 | .099 | 3.334 | .798 | 13.925 |
| Encounter for other preprocedural | .464 | .494 | .348 | 1.591 | .604 | 4.190 |
| examination (Z01.818) | | | | | | |
| Encounter for screening for | 1.587 | .599 | .008 | 4.889 | 1.512 | 15.807 |
| cardiovascular disorders (Z13.6) | | | | | | |
| Persons with potential health hazards | .285 | .287 | .321 | 1.329 | .757 | 2.334 |
| related to family and personal history | | | | | | |
| and certain conditions influencing | | | | | | |
| health status (Z77-Z99) | | | | | | |
| Planned Laminectomy | .479 | .265 | .071 | 1.614 | .960 | 2.714 |
| Planned Single Fusion | −.221 | .474 | .642 | .802 | .317 | 2.031 |
| Planned Pelvic Screw | −.540 | .467 | .247 | .583 | .233 | 1.454 |
| Planned Single Laminectomy | −.975 | .322 | .003 | .377 | .201 | .710 |
| Planned Single Interbody Fusion | −.439 | .506 | .386 | .645 | .239 | 1.738 |
| Constant | −1.583 | .889 | .075 | .205 | | |

Comparison of the predictive outcomes of the two models and their combination is illustrated in receiver operating characteristic curves in FIG. 5C. Table 4 reports the performance of the models on the training set, which can be viewed as the expected ceiling performance of the models.³³ The regression model, individually, achieved an area under the curve (AUC—an aggregate measure of performance across all possible classification thresholds) of 0.737 (training set AUC 0.808); a probability cutoff of 0.34 maximized the average outcome parameters (specificity 85.2%, sensitivity 49.2%, NPV 83.3%, and PPV 52.7%). The neural network achieved an AUC of 0.735 (training set AUC 0.753); a probability cutoff of 0.21 maximized the average outcome parameters (specificity 54.5%, sensitivity 84.7%, NPV 91.4%, and PPV 38.5%). At the same cutoff point of 0.5, no significant differences between the models were noted in sensitivity, specificity, NPV, or PPV for the testing set, as shown in Table 4. When applying four previously published models to our data set, each provided high specificities (94.4%-98.8%) but low sensitivities (6.2%-7.4%); see Table 4 for detailed results. The AUC was lower than in our models, with a range of 0.516-0.645.

TABLE 4
Prediction Outcomes

| | | | | | |
|---|---|---|---|---|---|
| Models | AUC | Specificity | Sensitivity | NPV | PPV |

Training Set (probability cutoff = 0.5)

| | | | | | |
|---|---|---|---|---|---|
| Regression | .808 | 95.4 | 42.0 | 81.9 | 76.8 |
| Neural Network | .753 | 94.4 | 26.0 | 77.8 | 62.9 |

Testing Set (probability cutoff = 0.5)

| | | | | | |
|---|---|---|---|---|---|
| Regression | .737 | 94.3 | 25.4 | 79.0 | 60.0 |
| Neural Network | .735 | 94.9 | 20.3 | 78.0 | 57.1 |
| Aiyer et al 2018 | .645 | 95.6 | 6.2 | 75.7 | 31.3 |
| Mormol et al 2020 | .638 | 94.4 | 7.4 | 75.7 | 30.0 |
| Nickerson et al 2016 | .559 | 98.8 | 6.2 | 76.3 | 62.5 |
| Altschul et al 2017 | .516 | 95.6 | 7.4 | 76.0 | 35.3 |

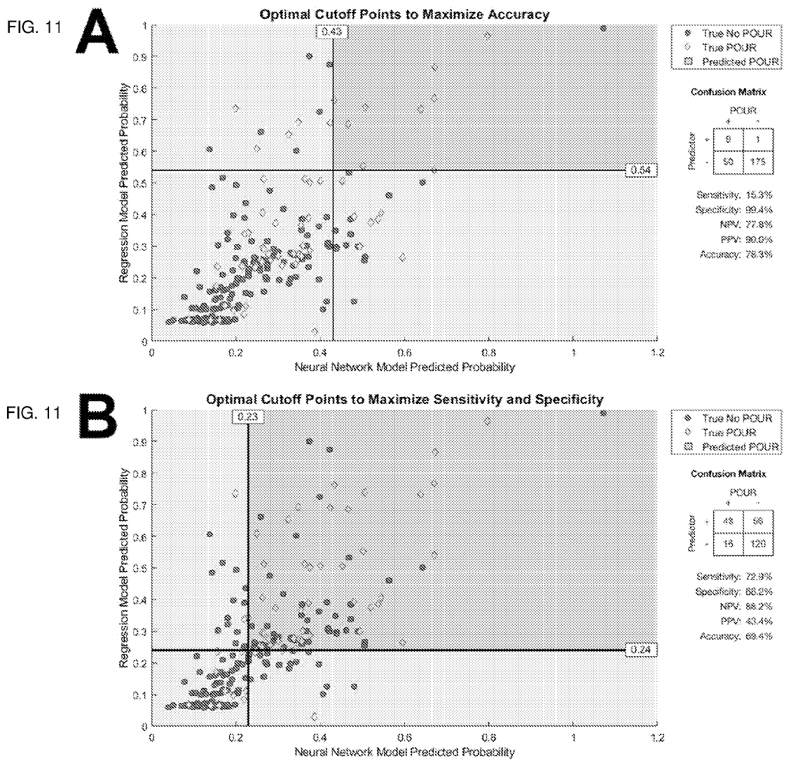

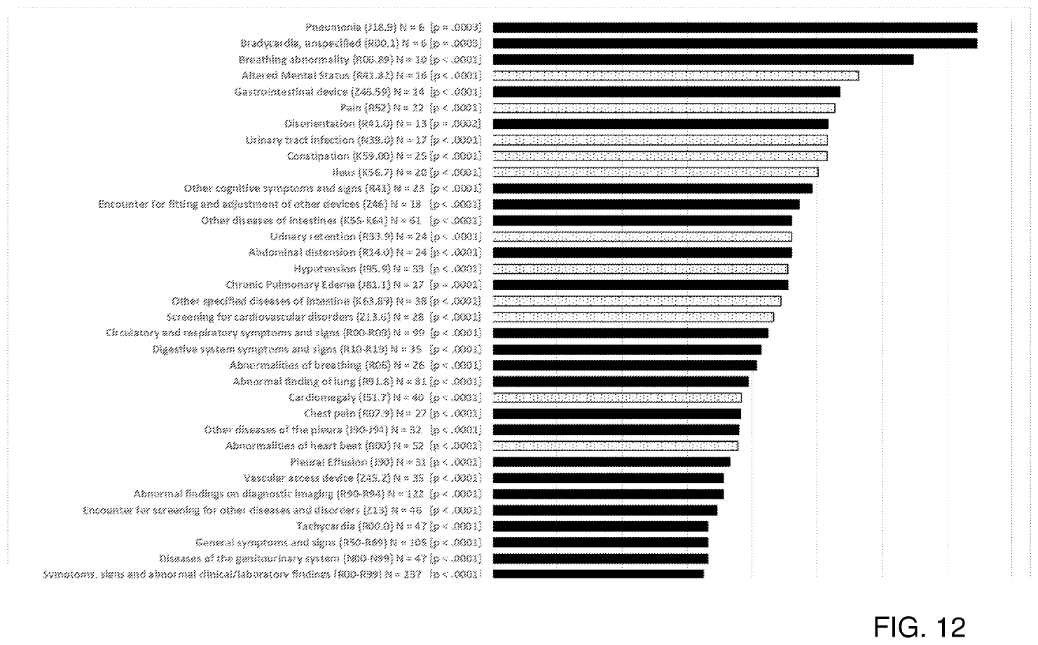

The stacking of the two models outperformed the individual models, with an AUC of 0.753. Optimal cutoff points for each model are demonstrated in Table 5. With the neural network alone and a cutoff of 0.14, sensitivity (96.6%) and NPV (96.1%) were simultaneously maximized, but with sacrifices in specificity (27.8%) and PPV (31.0%). With a regression model probability cutoff of 0.24 and a neural network cutoff of 0.23, sensitivity (72.9%) and specificity (68.2%) were simultaneously maximized, but with milder sacrifices in PPV (43.4%). With a regression model probability cutoff of 0.54 and a neural network cutoff of 0.43, all outcome parameters were simultaneously maximized (specificity 99.4%, sensitivity 15.3%, NPV 77.8%, and PPV 90.0%); however, sensitivity was severely sacrificed. FIG. 11 demonstrates graphically how the cutoff points are used to conjoin the models such that a positive prediction is derived when both the regression model predicted probability is greater than 0.54 and the neural network model predicted probability is greater than 0.43 as in FIG. 11A, or greater than 0.24 and 0.23, respectively, as in FIG. 11B. A spreadsheet (POUR Prediction Tool) is available in the Supplementary Materials J. Neurosurg Spine 36:32-41, 2021 (incorporated herein by reference), which assists in calculating the probability of developing POUR for each individual model and for the combined models.

TABLE 5
Model Cutoff Points to Maximize Prediction Outcomes
for Stacked (Combined) Model (AUC = 0.753)

| Regression | Neural Network | Specificity | Sensitivity | NPV | PPV |
|---|---|---|---|---|---|
| Cutoff | Cutoff | (%) | (%) | (%) | (%) |
| — | 0.14 | 27.8 | 96.6 | 96.1 | 31.0 |
| 0.54 | 0.43 | 99.4 | 15.3 | 77.8 | 90.0 |
| 0.24 | 0.23 | 72.9 | 68.2 | 88.2 | 43.4 |
| 0.54 | 0.43 | 99.4 | 15.3 | 77.8 | 90.0 |
| 0.54 | 0.43 | 99.4 | 15.3 | 77.8 | 90.0 |

### 5. ALTERNATIVES, DEVIATIONS AND MODIFICATIONS

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

### 6. REFERENCES

All the references listed here are hereby incorporated by reference as if fully set forth herein except for terminology inconsistent with that used herein.
1. Swann M C, Hoes K S, Aoun S G, McDonagh D L. Postoperative complications of spine surgery. Best Pract Res Clin Anaesthesiol. 2016; 30 (1): 103-120.
2. How many spinal fusions are performed each year in the United States? iData Research. Accessed Apr. 23, 2021. https://idataresearch.com/how-many-instrumented-spinalfusions-are-performed-each-year-in-the-united-states/
3. Altschul D, Kobets A, Nakhla J, et al. Postoperative urinary retention in patients undergoing elective spinal surgery. J Neurosurg Spine. 2017; 26(2): 229-234.
4. Baldini G, Bagry H. Aprikian A, Carli F. Postoperative urinary retention: anesthetic and perioperative considerations. Anesthesiology. 2009; 110(5): 1139-1157.
5. Strickland A R, Usmani M F, Camacho J E, et al. Evaluation of risk factors for postoperative urinary retention in elective thoracolumbar spinal fusion patients. Global Spine J. 2021; 11(3): 338-344.
6. Grass F, Slieker J, Frauche P, et al. Postoperative urinary retention in colorectal surgery within an enhanced recovery pathway. J Surg Res. 2017; 207:70-76.
7. Boulis N M, Mian F S, Rodriguez D, et al. Urinary retention following routine neurosurgical spine procedures. Surg Neurol. 2001; 55(1): 23-28.
8. Balderi T, Mistraletti G, D'Angelo E, Carli F. Incidence of postoperative urinary retention (POUR) after joint arthroplasty and management using ultrasound-guided bladder catheterization. Minerva Anestesiol. 2011; 77(11): 1050-1057.]
9. Cremins M, Vellanky S, McCann G, et al. Considering healthcare value and associated risk factors with postoperative urinary retention after elective laminectomy. Spine J. 2020; 20(5): 701-707.
10. Garg D, Agarwal A. Comment on “Early presentation of urinary retention in multiple system atrophy: can the disease begin in the sacral spinal cord?”. J Neurol. 2020; 267(3): 665.
11. Agrawal K. Majhi S, Garg R. Post-operative urinary retention: review of literature. World J Anesthesiol. 2019; 8(1): 1-12.
12. Mouchtouris N, Hines K, Fitchett E M, et al. Cost of postoperative urinary retention after elective spine surgery: significant variation by surgeon and department. Neurosurgery. 2020; 67(suppl 1): nyaa447_115.
13. Golubovsky J L, Ilyas H, Chen J, et al. Risk factors and associated complications for postoperative urinary retention after lumbar surgery for lumbar spinal stenosis. Spine J. 2018; 18(9): 1533-1539.
14. Hospital adjusted expenses per inpatient day by ownership. KFF. Accessed Apr. 23, 2021. https://www.kff.org/health-costs/state-indicator/expenses-per-inpatient-day-byownership/
15. Sullivan N M, Sutter V L, Mims M M, et al. Clinical aspects of bacteremia after manipulation of the genitourinary tract. J Infect Dis. 1973; 127(1): 49-55.
16. Estimating the additional hospital inpatient cost and mortality associated with selected hospital-acquired conditions. Agency for Health Research and Quality. Accessed Apr. 23, 2021. https://www.ahrq.gov/hai/pfp/haccost2017-results.html
17. Mormol J D, Basques B A, Harada G K, et al. Risk factors associated with development of urinary retention following posterior lumbar spinal fusion: special attention to the use of glycopyrrolate in anesthesia reversal. Spine (Phila Pa 1976). 2021; 46(2): E133-E138.
18. Aiyer S N, Kumar A. Shetty A P, et al. Factors influencing postoperative urinary retention following elective posterior lumbar spine surgery: a prospective study. Asian Spine J. 2018; 12(6): 1100-1105.
19. Nickerson P, Tighe P, Shickel B, Rashidi P. Deep neural network architectures for forecasting analgesic response. Annu Int Conf IEEE Eng Med Biol Soc. 2016; 2016:2966-2969.
20. Balabaud L, Pitel S, Caux I. et al. Lumbar spine surgery in patients 80 years of age or older: morbidity and mortality. Eur J Orthop Surg Traumatol. 2015; 25(suppl 1): S205-S212.
21. Knight B A, Bayne A P, Zusman N, et al. Postoperative management factors affect urinary retention following posterior spinal fusion for adolescent idiopathic scoliosis. Spine Deform. 2020; 8(4): 703-709.
22. Petros J G, Bradley T M. Factors influencing postoperative urinary retention in patients undergoing surgery for benign anorectal disease. Am J Surg. 1990; 159(4): 374-376.
23. Petros J G, Rimm E B, Robillard R J, Argy O. Factors influencing postoperative urinary retention in patients undergoing elective inguinal herniorrhaphy. Am J Surg. 1991; 161(4): 431-434.
24. Petros J G, Rimm E B, Robillard R J. Factors influencing urinary tract retention after elective open cholecystectomy. Surg Gynecol Obstet. 1992; 174(6): 497-500.
25. Petros J G, Mallen J K, Howe K, et al. Patient-controlled analgesia and postoperative urinary retention after open appendectomy. Surg Gynecol Obstet. 1993; 177(2): 172-175.
26. Petros J G, Alameddine F, Testa E, et al. Patient-controlled analgesia and postoperative urinary retention after hysterectomy for benign disease. J Am Coll Surg. 1994; 179(6): 663-667.
27. Faas C L, Acosta F J, Campbell M D R, et al. The effects of spinal anesthesia vs epidural anesthesia on 3 potential postoperative complications: pain, urinary retention, and mobility following inguinal herniorrhaphy. AANA J. 2002; 70(6): 441-447.
28. Larsen J, Goutte C. On optimal data split for generalization estimation and model selection. In: Neural Networks for Signal Processing IX: Proceedings of the 1999 IEEE Signal Processing Society Workshop. IEEE; 1999:225-234.
29. Draelos R. Best use of train/val/test splits, with tips for medical data. Glass Box. Published Sep. 15, 2019. Accessed Apr. 23, 2021. https://glassboxmedicine.com/2019/09/15/best-use-of-train-val-test-splits-with-tips-for-medical-data/
30. Bursac Z. Gauss C H, Williams D K, Hosmer D W. Purposeful selection of variables in logistic regression. Source Code Biol Med. 2008; 3:17.
31. Harrell F E Jr. Binary logistic regression. In: Harrell F E Jr, ed. Regression Modeling Strategies: With Applications to Linear Models, Logistic and Ordinal Regression, and Survival Analysis. 2nd ed. Springer Series in Statistics. Springer International Publishing; 2015:219-274.
32. Bisong E. Ensemble methods. In: Bisong E, ed. Building Machine Learning and Deep Learning Models on Google Cloud Platform: A Comprehensive Guide for Beginners. Apress; 2019: 269-286.
33. Bisong E. Principles of learning. In: Bisong E, ed. Building Machine Learning and Deep Learning Models on Google Cloud Platform: A Comprehensive Guide for Beginners. Apress; 2019: 171-197.
34. Meddings J, Skolarus T A, Fowler K E, et al. Michigan Appropriate Perioperative (MAP) criteria for urinary catheter use in common general and orthopaedic surgeries: results obtained using the RAND/UCLA Appropriateness Method. BMJ Qual Saf. 2019; 28(1): 56-66.
35. Hoke N, Bradway C. A clinical nurse specialist-directed initiative to reduce postoperative urinary retention in spinal surgery patients. Am J Nurs. 2016; 116(8): 47-52.
36. Turan A, Karamanlioğlu B, Memiş D, et al. Analgesic effects of gabapentin after spinal surgery. Anesthesiology. 2004; 100(4): 935-938.
37. Madani A H, Aval H B, Mokhtari G, et al. Effectiveness of tamsulosin in prevention of post-operative urinary retention: a randomized double-blind placebo-controlled study. Int Braz J Urol. 2014; 40(1): 30-36.
38. Fan F, Xiong J, Li M, Wang G. On interpretability of artificial neural networks: a survey. ArXiv. Preprint posted online Nov. 30, 2020. http://arxiv.org/abs/2001.02522
39. Pavlyshenko B. Using stacking approaches for machine learning models. In: 2018. IEEE Second International Conference on Data Stream Mining Processing (DSMP). IEEE; 2018: 255-258.
40. Mandrekar J N. Receiver operating characteristic curve in diagnostic test assessment. J Thorac Oncol. 2010; 5(9): 1315-1316.

What is claimed is:

1. A method for treating a subject undergoing surgery, the method comprising:
obtaining, on a processor, first data for a subject undergoing surgery, wherein the first data indicates demographic information for the subject or medical history for the subject or surgery information about the spinal surgery, or some combination;
generating, on the processor, neural network output data that indicates a first probability for the subject developing post-operative outcome by inputting the first data into an input layer of a neural network trained with training data that indicates, for a retrospective plurality of prior patients of spinal surgery, demographic information for the retrospective plurality or medical history for the retrospective plurality or surgery information about spinal surgeries for the retrospective plurality, or some combination, and post-operative outcomes for the retrospective plurality;
sending from the processor a signal that indicates a post-operative outcome classification for the subject based at least in part on the neural network output data; and
treating the subject based at least in part on the signal.

2. The method as recited in claim 1, wherein the signal indicates that the subject should be treated for the post-operative outcome when the first probability exceeds a first cutoff value.

3. The method as recited in claim 1, wherein the first data includes values for over 200 parameters.

4. The method as recited in claim 3, wherein said inputting the first data into the input layer of the neural network further comprises scaling the first data with a scaling factor for each parameter such that all values of that parameter for the training data lie in a range from −1 to 1 inclusive.

5. The method as recited in claim 1, wherein the neural network comprises two hidden layers, each hidden layer fully connected to a preceding layer and each hidden layer using a sigmoid activation function.

6. The method as recited in claim 5, wherein a first hidden layer of the two hidden layers is fully connected to the input layer and the first hidden layer comprises a first number nodes in a range from 20 to 80.

7. The method as recited in claim 6, wherein a second hidden layer of the two hidden layers is fully connected to the first hidden layer and the second hidden layer comprises a second number nodes in a range from 10 to 40.

8. The method as recited in claim 1, wherein an output layer of the neural network comprises one output node that indicates the first probability and uses an identity activation function and wherein the output layer uses a sum of squares error function during training.

9. The method as recited in claim 1, wherein:
the method further comprises generating, on the processor, multiple regression output data that indicates a second probability for the subject developing the post-operative outcome by inputting a small subset of the first data into an input layer of a multiple regression trained with a corresponding subset of the training data; and
the post-operative outcome classification for the subject is further based at least in part on the multiple regression output data.

10. The method as recited in claim 9, wherein the signal indicates that the subject should be treated for the post-operative outcome when the first probability exceeds a first cutoff value or when the second probability exceeds a second cutoff value.

11. The method as recited in claim 9, wherein the signal indicates that the subject should be treated for the post-operative outcome when the first probability exceeds a first cutoff value and when the second probability exceeds a second cutoff value.

12. The method as recited in claim 9, wherein the small subset includes values for fewer than 50 parameters.

13. The method as recited in claim 9, wherein the small subset includes only subset parameters of the first data, wherein the subset parameters are correlated with the post-operative outcomes for the training set with a p value less than threshold significance level.

14. The method as recited in claim 13, wherein the threshold significance level is a p value less than 0.05.

15. The method as recited in claim 1, wherein said treating the subject comprises administering a the post-operative outcome intervention therapy.

16. The method as recited in claim 1, wherein the post-operative outcome is post-operative urinary retention (POUR) outcome and the surgery is spinal surgery.

17. The method as recited in claim 16, wherein the surgery is lower spinal surgery.

18. A non-transitory computer-readable medium carrying one or more sequences of instructions, wherein execution of the one or more sequences of instructions by one or more processors causes the one or more processors to perform at least:
obtaining, on a processor, first data for a subject undergoing surgery, wherein the first data indicates demographic information for the subject or medical history for the subject or surgery information about the surgery, or some combination;
generating, on the processor, neural network output data that indicates a first probability for the subject developing post-operative outcome by inputting the first data into an input layer of a neural network trained with training data that indicates, for a retrospective plurality of prior patients of spinal surgery, demographic information for the retrospective plurality or medical history for the retrospective plurality or surgery information about spinal surgeries for the retrospective plurality, or some combination, and post-operative outcomes for the retrospective plurality; and
sending from the processor a signal that indicates a post-operative outcome classification for the subject based at least in part on the neural network output data, wherein treatment of the subject is based at least in part on the signal.

19. An apparatus comprising:
at least one processor; and
at least one memory including one or more sequences of instructions,
the at least one memory and the one or more sequences of instructions configured to, with the at least one processor, cause the apparatus to perform at least the following,
obtain, on a processor, first data for a subject undergoing surgery, wherein the first data indicates demographic information for the subject or medical history for the subject or surgery information about the surgery, or some combination;
generate, on the processor, neural network output data that indicates a first probability for the subject developing post-operative outcome by inputting the first data into an input layer of a neural network trained with training data that indicates, for a retrospective plurality of prior patients of spinal surgery, demographic information for the retrospective plurality or medical history for the retrospective plurality or surgery information about spinal surgeries for the retrospective plurality, or some combination, and post-operative outcomes for the retrospective plurality; and
send from the processor a signal that indicates a post-operative outcome classification for the subject based at least in part on the neural network output data, wherein treatment of the subject is based at least in part on the signal.
